(19) United States
(12) Patent Application Publication
(10) Pub. No.: US 2025/0270199 A1
(43) Pub. Date: Aug. 28, 2025

(54) THIAZOLE DERIVATIVES AND METHODS OF USING THE SAME

(71) Applicant: Arkansas State University - Jonesboro, State University, AR (US)

(72) Inventor: Mohammad Abrar ALAM, Jonesboro, AR (US)

(21) Appl. No.: 18/853,013

(22) PCT Filed: Mar. 31, 2023
(86) PCT No.: PCT/US2023/017103

§ 371 Date: Sep. 30, 2024

Related U.S. Application Data
(60) Provisional application No. 63/326,114, filed on Mar. 31, 2022.

(51) Int. Cl. C07D 417/04; A61K 31/426; A61K 31/427; A61K 31/428; A61K 31/4439; A61P 31/04; C07D 277/24; C07D 277/30; C07D 277/42; C07D 277/66; C07D 277/84

(52) U.S. Cl. CPC C07D 417/04; A61K 31/426; A61K 31/427; A61K 31/428; A61K 31/4439; A61P 31/04; C07D 277/24; C07D 277/30; C07D 277/42; C07D 277/66; C07D 277/84

(57) ABSTRACT

Disclosed herein am thiazolylphenol compounds 1 and methods of using the same for the treatment of subjects in need of a treatment for an infection by a microbe.

### CROSS-REFERENCE TO RELATED APPLICATIONS

This application claims benefit of priority to U.S. Patent Application No. 63/326,114, filed Mar. 31, 2022, the contents of which is incorporated by reference in its entirety.

### FIELD OF THE INVENTION

The disclosed technology is generally directed to thiazole derivatives. More particularly the technology is directed to thiazole derivatives for antimicrobial applications.

### BACKGROUND OF THE INVENTION

Antibiotic resistance is one of the world's urgent public health problems, which affects people at any stage of life, healthcare system, veterinary and agricultural industries. More than 2.8 million people are infected with antibiotic-resistance bacteria or fungi and more than 35,000 people die of these infections in the United States alone. Many modern healthcare advances such as joint replacements, organ transplants, cancer therapy, and the treatment of chronic diseases like diabetes, arthritis, and asthma are dependent on the ability to fight infections using antibiotics.¹² Staphylococcus aureus is found in about 30% people's nares. This bacterium can cause sepsis, pneumonia, endocarditis, and osteomyelitis. The treatment of S. aureus infections often becomes challenging due to its ability to develop resistance against approved antibiotics. Based on S. aureus sensitivity to antibiotics, this bacterium is known as different germs including: methicillin-sensitive S. aureus (MSSA), methicillin-resistant S. aureus (MRSA), vancomycin-intermediate S. aureus (VISA), and vancomycin-resistant S. aureus (VRSA). Penicillin and daptomycin resistant S. aureus have also been reported over the years. Although MRSA is most common drug-resistant S. aureus strain, any strain of this bacterium could be dangerous.¹²⁻¹³ As a result, there is a need for new antimicrobial compounds and compositions.

### BRIEF SUMMARY OF THE INVENTION

One aspect of the technology provides a thiazolylphenol compound, or a pharmaceutically acceptable salt thereof, wherein the phenol may be optionally substituted with a second hydroxyl group and wherein the thiazolyl is substituted with one or more substituents independently selected from: a phenylamino substituted at one or more positions with a halo, an alkyl, an alkoxyl, a haloalkyl, or any combination thereof, an aryl optionally substituted at one or more positions with a halogen, an alkyl, a haloalkyl, a hydroxyl, an alkoxyl, cyano, oxo, carboxyl, acetamide, or any combination thereof, an alkyl optionally substituted with a halo, a heteroaryl, and a biaryl or two substituents forming a fused carbocyclyl with the thiozolyl.

In some aspects, the thiazolyl and phenol may be covalently bound at the 4-position or 2-position of the thiazolyl.

In some aspects, the phenol may be

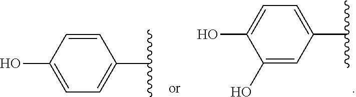 [SMILES: *c1ccc(O)cc1 ; *c1ccc(O)c(O)c1]

One aspect of the technology provides for thiazolylphenol compounds of formula

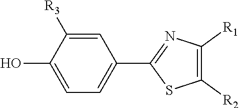

or a pharmaceutically acceptable salt, thereof, wherein R₃ may be selected from a hydroxyl or hydrogen, R₁ and R₂ may be independently selected from hydrogen, the substituted or unsubstituted alkyl, the substituted or unsubstituted aryl, the heteroaryl, and the biaryl and both R₁ and R₂ are not hydrogen or R₁ and R₂ together form a fused carbocyclyl with the thiozolyl.

Another aspect of the technology provides for thiazolylphenol compounds of formula

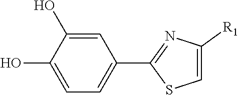

or a pharmaceutically acceptable salt thereof, wherein R₁ may be selected from the substituted or unsubstituted alkyl, the substituted or unsubstituted aryl, the heteroaryl, and the biaryl. In some aspects, R₁ may be selected from the substituted or unsubstituted aryl.

Another aspect of the technology provides for thiazolylphenol compounds of formula

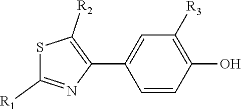

or a pharmaceutically acceptable salt thereof, wherein R₃ may be hydrogen or hydroxyl, wherein R₂ may be hydrogen, and wherein R₁ may be selected from the substitute phenylamino and the substituted or unsubstituted aryl. In some aspects, R₁ may be the substituted phenylamino. In some aspects, R₁ may be the substituted or unsubstituted aryl.

Another aspect provides for pharmaceutical compositions comprising any of the thiazolylphenol compounds disclosed herein and a pharmaceutically acceptable excipient, carrier, or diluent. The pharmaceutical composition may be comprising an effective amount of any of the compounds disclosed herein.

Another aspect provides for is a method for the treatment of a subject in need of a treatment for an infection by a microbe. The method may comprise administering an effective amount of any of the compounds described herein or a pharmaceutical composition comprising the effective amount of the compound to the subject.

Another aspect provided for is a method for inhibiting growth or proliferation or killing a microbe. The method may comprise contacting the microbe with an effective amount of any of the compounds described herein.

In some embodiments, the microbe is antimicrobial resistant.

In some embodiments, the microbe is a persister.

In some embodiments, the microbe is a Gram-positive bacterium. Exemplary Gram-positive bacterium includes, without limitation. S. aureus, S. epidermidis, B. sublilis, E. faecalis, or E. faecium. In some embodiments, the Gram-positive bacterium is a methicillin-resistant S. aureus.

These and other aspects of the technology will be further described herein.

### DETAILED DESCRIPTION OF THE INVENTION

Disclosed herein are thiazole derivatives and methods of using the same. As demonstrated in the Examples, the presently disclosed compounds are effective antimicrobials for killing or inhibiting the growth or proliferation of microbes, such as Gram-positive and -negative bacteria.

A notable advantage of the compounds disclosed herein is that they effective against antimicrobial-resistant and persister strains. The compounds also demonstrated greater potency than front-line antibiotics, such as vancomycin and gentamicin, and are non-toxic in human cell lines.

### Compounds

One aspect of the technology provides a thiazolylphenol compound, or a pharmaceutically acceptable salt thereof. The thiazolylphenol may be represented by formula

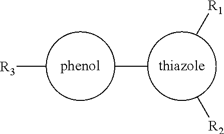

where the phenol and thiazolyl rings are covalently bonded by a carbon-carbon bond and the
thiazolyl ring comprises at least one non-hydrogen substituent, R₁. The thiazolylphenol may optionally comprise one or more additional substituents, R₂ and R₃, on the thiazolyl and phenol rings, respectively. The circular representation of the rings indicates that the phenol and thiazolyl rings may be bonded at different carbon positions on either ring. In some aspects, the thiazolyl and phenol may be covalently bound at the 4-position of the thiazolyl. In other aspects, the thiazolyl and phenol may be covalently bound at the 2-position of the thiazolyl.

The term “phenol” as used herein refers to a compound with one or more hydroxyl groups linked directly to a phenyl ring. In some aspects, the phenol comprises one hydroxyl. In some embodiments, the phenol is

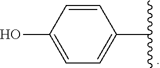 [SMILES: *c1ccc(O)cc1]

In other aspects, the phenol may be optionally substituted with a second hydroxyl group. In other aspects, the phenol comprises two hydroxyl groups, i.e., a benzenediol. The two hydroxyl groups may be at adjacent ring positions. When the two hydroxyl groups are at adjacent ring positions, the phenol may be characterized as a catechol (i.e., catechol-substituted thiazole derivatives). In some embodiments, the phenol is

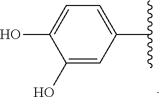 [SMILES: *c1ccc(O)c(O)c1]

The thiazolyl is substituted with one or more substituents. In some embodiments, the thiazolyl is substituted with one or more substituents independently selected from a phenylamino, an aryl, an alkyl, a heteroaryl, or a biaryl and each of the substituents may be optionally substituted. The optional substituents may be a halogen, an alkyl, a haloalkyl, a hydroxyl, an alkoxyl, cyano, oxo, carboxyl, acetamide, or any combination thereof. Exemplary substituents bound to the thiazolyl include, without limitation, a phenylamino substituted at one or more positions with a halo, an alkyl, an alkoxyl, a haloalkyl, or any combination thereof; an aryl optionally substituted at one or more positions with a halogen, an alkyl, a haloalkyl, a hydroxyl, an alkoxyl, cyano, oxo, carboxyl, acetamide, or any combination thereof, an alkyl optionally substituted with a halo; heteroaryl optionally substituted with oxo, a biaryl, or any combination thereof.

In some embodiments, the thiazolyl is substituted with a phenylamino substituted at one or more positions with a halo (e.g., chloro or fluoro), an alkyl (e.g., methyl), an alkoxyl (e.g., methoxy), a haloalkyl (e.g., trifluoromethyl), or any combination thereof.

In some embodiments, the thiazolyl is substituted with an aryl (e.g., phenyl or naphthyl) optionally substituted at one or more positions with a halogen (e.g., chloro or fluoro), an alkyl (e.g., methyl), a haloalkyl (e.g., trifluoromethyl), a hydroxyl, an alkoxyl (e.g., methoxy), cyano, oxo, carboxyl. acetamide, or any combination thereof.

In some embodiments, the thiazolyl is substituted with an alkyl optionally substituted with a halo (e.g., chloro or fluoro).

In some embodiments, the thiazolyl is substituted with a heteroaryl (e.g., pyridinyl or coumarinyl) optionally substituted with oxo. Examples include, pyridin-4-yl or coumarin-3-yl.

In some embodiments, the thiazolyl is substituted with a biaryl.

Examples of substituents bound to the thiazolyl include methyl, trifluoroethyl,

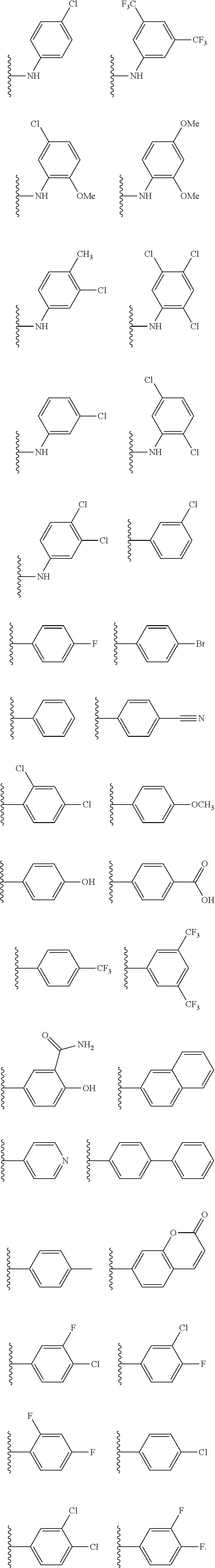

In some embodiments, the thiazolyl is substituted with two substituents that together form a fused carbocyclyl with the thiozolyl.

One aspect of the technology provides for thiazolylphenol compounds of Formula I

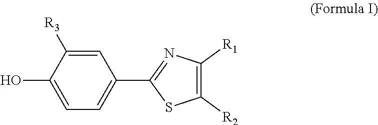

or a pharmaceutically acceptable salt thereof.

R₃ of Formula I may be selected from a hydroxyl or hydrogen. In some aspects, R₃ is hydrogen. In other aspects, R₁ is hydroxyl.

In some embodiments of Formula I, R₁ and R₂ may be independently selected from hydrogen, an alkyl, an aryl, a heteroaryl, and a biaryl provided that both R₁ and R₂ are not hydrogen. In some embodiments, R₂ is hydrogen or an alkyl. In some embodiments, R₂ is hydrogen or an alkyl and R₁ is selected from an aryl, a heteroaryl, and a biaryl. R₁ and/or R₂ may be optionally substituted at one or more positions with halogen, haloalkyl, hydroxyl, alkoxyl, cyano, oxo, carboxyl, or acetamide. Exemplary R₁ and R₂ are provided by the Examples.

In other embodiments of Formula I, R₁ and R₁ may together form a fused carbocyclyl with the thiozolyl. Exemplary R₁ and R₂ are provided the the Examples.

One aspect of the technology provides for thiazolylphenol compounds of Formula II

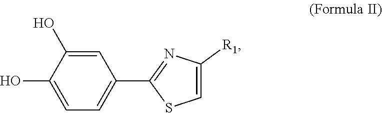

or a pharmaceutically acceptable salt thereof. R₁ may be selected from an alkyl, an aryl, a heteroaryl, and a biaryl. R₁ may be optionally substituted at one or more positions with halogen, haloalkyl, hydroxyl, alkoxyl, cyano, oxo, carboxyl, or acetamide. Exemplary R₁ and R₂ are provided by the Examples.

One aspect of the technology provides for thiazolylphenol compounds of Formula (III)

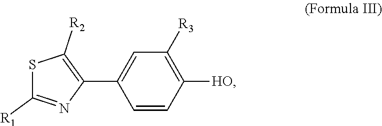

or a pharmaceutically acceptable salt thereof.

R₃ of Formula III may be selected from a hydroxyl or hydrogen. In some aspects, R₃ is hydrogen. In other aspects, R₃ is hydroxyl.

R₂ of Formula III may be hydrogen.

R₁ of Formula III may be selected from a phenylamino and an aryl. In some aspects, R₁ is the substituted phenylamino. In some aspects, R₁ is the aryl. R₁ may be optionally substituted at one or more positions with halogen, haloalkyl, hydroxyl, alkoxyl, cyano, oxo, carboxyl, or acetamide. Exemplary R₁ are provided by the Examples.

Exemplary thiazolyphenol compounds include:
4-(4-(trifluoromethyl)thiazol-2-yl)benzene-1,2-diol,
4-(4-phenylthiazol-2-yl)benzene-1,2-diol,
4-(4-(3-chlorophenyl)thiazol-2-yl)benzene-1,2-diol,
4-(4-(4-fluorophenyl)thiazol-2-yl)benzene-1,2-dial,
4-(4-(4-bromophenyl)thiazol-2-yl)benzene-1,2-dial,
4-(4-(2,4-dichlorophenyl)thiazol-2-yl)benzene-1,2-diol,
4-(4-(4-methoxyphenyl)thiazol-2-yl)benzene-1,2-dial,
4-(4-(4-hydroxyphenyl)thiazol-2-yl)benzene-1,2-diol,
4-(4-(4-(trifluoromethyl)phenyl)thiazol-2-yl)benzene-1,2-diol,
4-(4-(3,5-bis(trifluoromethyl)phenyl)thiazol-2-yl)benzene-1,2-diol,
4-(4-(pyridin-4-yl)thiazol-2-yl)benzene-1,2-dial,
4-(2-(3,4-dihydroxyphenyl)thiazol-4-yl)benzonitrile,
4-(2-(3,4-dihydroxyphenyl)thiazol-4-yl)benzoic acid,
4-(4-(naphthalen-2-yl)thiazol-2-yl)benzene-1,2-diol,
4-(4[1,1′-biphenyl]-4-yl)thiazol-2-yl)benzene-1,2-diol,
5-(2-(3,4-di hydroxyphenyl)thiazol-4-yl)-2-hydroxybenzamide,
3-(2-(3,4-dihydroxyphenyl)thiazol-4-yl)-2H-chromen-2-one,
4-(4-(p-tolyl)thiazol-2-yl)benzene-1,2-diol,
4-(4-(4-fluoro-3-(trifluoromethyl)phenyl)thiazol-2-yl)benzene-1,2-diol,
4-(4-(4-fluorophenyl)-5-methylthiazol-2-yl)benzene-1,2-diol,
4-(4-(3-chloro-4-fluorophenyl)thiazol-2-yl)benzene-1,2-diol,
4-(5-methyl-4-phenylthiazol-2-yl)benzene-1,2-diol,
4-(5-methyl-4-(p-tolyl)thiazol-2-yl)benzene-1,2-diol,
4-(4-(3-chlorophenyl)-5-methylthiazol-2-yl)benzene-1,2-diol,
4-(4,5-diphenylthiazol-2-yl)benzene-1,2-diol,
4-(4-(2,4-difluorophenyl)thiazol-2-yl)benzene-1,2-diol,
4-(4-(4-chlorophenyl)thiazol-2-yl)benzene-1,2-diol,
4-(4,5,6,7-tetrahydrobenzo[d]thiazol-2-yl)benzene-1,2-diol,
4-(5,6-dihydro-4H-cyclopenta[d]thiazol-2-yl)benzene-1,2-diol,
4-(4,5-di hydronaphtho[1,2-d]thiazol-2-yl)benzene-1,2-diol,
4-(4-(3,4-dichlorophenyl)thiazol-2-yl)benzene-1,2-diol,
4-(4-phenyl thiazol-2-yl)phenol,
4-(4-(4-bromophenyl)thiazol-2-yl)phenol,
4-(4-(2,4-dichlorophenyl)thiazol-2-yl)phenol,
4-(4-(naphthalen-2-yl)thiazol-2-yl)phenol,
4-(4-(3,5-bis(trifluoromethyl)phenyl)thiazol-2-yl)phenol,
4-(2-(4-hydroxyphenyl)thiazol-4-yl)benzoic acid,
4-(4-(3-chlorophenyl)thiazol-2-yl)phenol,
4-(4-(4-(trifluoromethyl)phenyl)thiazol 2-yl)phenol,
4,4′-(thiazole-2,4-diyl)diphenol,
4-(4-(3,4-dichlorophenyl)thiazol-2-yl)phenol,
4-(2-(4-bromophenyl)thiazol-4-yl)phenol,
4-(2-(3,5-dichlorophenyl)thiazol-4-yl)phenol,
4-(2-(4-(trifluoromethyl)phenyl)thiazol-4-yl)phenol,
4-(2-(4-bromophenyl)thiazol-4-yl)benzene-1,2-diol,
4-(2-(3,5-dichlorophenyl)thiazol-4-yl)benzene-1,2-diol,
4-{2-[4-(trifluoromethyl)phenyl]-1,3-thiazol-4-yl}benzene-1,2-diol4-(2-(4-chlorophenyl)thiazol-4-yl)phenol,
4-(2-(4-chlorophenyl(thiazol-4-yl)benzene-1,2-diol,
4-(2-(4-fluorophenyl)thiazol-4-yl)phenol,
4-(2-(4-fluorophenyl)thiazol-4-yl)benzene-1,2-diol,
4-(2-(3,4-difluorophenyl)thiazol-4-yl)phenol,
4-(2-(3,4-difluorophenyl)thiazol-4-yl)benzene-1,2-diol,
4-(2-(3-chlorophenyl)thiazol-4-yl)phenol,
4-(2-(3-chlorophenyl)thiazol-4-yl)benzene-1,2-diol,
4-(2-((4-chlorophenyl)amino)thiazol-4-yl)benzene-1,2-diol,
4-(2-((4-chlorophenyl)amino)thiazol-4-yl)phenol,
4-(2-((3,5-bis(trifluoromethyl)phenyl)amino)thiazol-4-yl)benzene-1,2-diol,
4-(2-((3,5-bis(trifluoromethyl)phenyl)amino)thiazol-4-yl)phenol,
4-(2-((3-methoxyphenyl)amino)thiazol-4-yl)benzene-1,2-diol,
4-(2-((5-chloro-2-methoxyphenyl)amino)thiazol-4-yl)phenol,
4-(2-((2,4-dimethoxyphenyl)amino)thiazol-4-yl)benzene-1,2-diol,
4-(2-((2,4-dimethoxyphenyl)amino)thiazol-4-yl)phenol,
4-(2-(3,4-dichlorophenyl)thiazol-4-yl)benzene-1,2-diol,
4-(2-(3,4-dichlorophenyl)thiazol-4-yl)phenol,
4-(2-((3-chloro-4-methylphenyl)amino)thiazol-4-yl)benzene-1,2-diol,
4-(2-((3-chloro-4-methylphenyl)amino)thiazol-4-yl)phenol,
4-(2-((2,4,5-trichlorophenvl)amino)thiazol-4-yl)benzene-1,2-diol,
4-(2-((2,4,5-trichlorophenyl)amino)thiazol-4-yl)phenol,
4-(2-((2,3-dichlorophenyl)amino)thiazol-4-yl)benzene-1,2-diol,
4-(2-((2,3-dichlorophenyl)amino)thiazol-4-yl)phenol,
4-(2-((2,5-dichlorophenyl)amino)thiazol-4-yl)benzene-1,2-diol,
4-(2-((2,5-dichlorophenyl)amino)thiazol-4-yl)phenol,
4-(2-((3,4-dichlorophenyl)amino)thiazol-4-yl)benzene-1,2-diol, and
4-(2-((3,4-dichlorophenyl)amino)thiazol-4-yl)phenol.

The thiazolylphenol compounds disclosed herein may be prepared by the reaction schemes provided in the Examples.

As used herein, an asterick “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, 1-6, or 1-4 carbon atoms, referred to herein as C₁-C₁₂ alkyl, C₁-C₁₀-alkyl, C₁-C₆-alkyl, C₁-C₄-alkyl respectively.

The term “alkylene” refers to a diradical of an alkyl group. An exemplary alkylene group is —CH₂CH₂—.

The term “haloalkyl” refers to an alkyl group that is substituted with at least one halogen.

For example, —CH₂F, —CH₂F₂, —CF₃, —CH₂CF₃, —CF₂CF₃, and the like

The term “heteroalkyl” as used herein refers to an “alkyl” group in which at least one carbon atom has been replaced with a heteroatom (e.g., an O, N, or S atom). One type of heteroalkyl group is an “alkoxyl” group

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₁-C₁₂-alkynyl, C₂-C₁₀-alkynyl, and C₁-C₆-alkynyl, respectively

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number oring carbon atoms. For example, the partially unsaturated carbocyclyl may contain 5-14, 5-12, 5-8, or 5-6 ring carbon atoms, and accordingly be referred to as a C₅-C₄, C₅-C₁₂, C₅-C₈, or C₅-C₆ membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The teen “aryl” is art-recognized and refers to a carbocyclic aromatic group. Representative aryl groups include phenyl, naphthyl, anthracenyl, and the like. The term “aryl” includes polycyclic ring systems having two or more carbocylic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

An “epoxide” is a cyclic ether with a three-atom ring typically include two carbon atoms and whose shape approximates an isosceles triangle. Epoxides can be formed by oxidation of a double bound where the carbon atoms of the double bond form an epoxide with an oxygen atom.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —R¹C(O)N(R²)—, —R¹C(O)N(R²) R³—, —C(O)N R² R³, or —C(O)NH₂, wherein R¹, R² and R³ are independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The term “phenylamino” as used herein refers to a group of formula

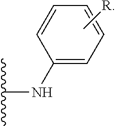

The phenyl ring may be substituted at one or more positions. Exemplary substituents include, halo (e.g., chloro), alkyl (e.g., methyl), alkoxyl (e.g., methoxy), or haloalkyl (e.g., trifluoromethyl).

The term “carbocyclyl” as used herein refers to a ring structure composed of carbon atoms. The carbocyclyl may be saturated, partially unsaturated, or unsaturated, A “fused carbocyclyl” as used herein refers to a carbocyclyl having two, and only two, atoms in common with at least one other ring.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Compositions comprising substantially purified stereoisomers, epimers, or enantiomers, or analogs or derivatives thereof are contemplated herein (e.g., a composition comprising at least about 90%, 95%, or 99% pure stereoisomer, epimer, or enantiomer.)

### Pharmaceutical Compositions

The compounds disclosed herein may be formulated as pharmaceutical compositions that include: an effective amount of one or more compounds and one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carver. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods of disclosed herein may be administered as a single compound or a combination of compounds.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Pharmaceutically acceptable esters and amides of the compounds can also be employed in the compositions and methods disclosed herein.

In addition, the methods disclosed herein may be practiced using solvate forms of the compounds or salts, esters, and/or amides, thereof. Solvate forms may include ethanol solvates, hydrates, and the like.

### Methods

Methods for treating subjects with the compounds disclosed herein are provided. Suitably the method for treating a subject comprises administering to the subject an effective amount of one or more of the compounds disclosed herein or a pharmaceutical composition comprising the effective amount of one or more of the compounds disclosed herein. As used herein, a “subject” may be interchangeable with “patient” or “individual” and means an animal, which may be a human or non-human animal, in need of treatment, A “subject in need of treatment” may include a subject having a disease, disorder, or condition that is responsive to therapy with one or more of the compounds disclosed herein. In some embodiments, the subject is responsive to therapy with one or more of the compounds disclosed herein in combination with one or more additional therapeutic agents. For example, a “subject in need of treatment” may include a subject in need of treatment for a microbial infection. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

In some embodiments, the subject has a microbial infection and may show symptoms associated therewith. Symptoms associated with microbial infections can be varied depending on the location and severity of the infection. In some instances, the infection is located in or on the skin or an inner organ, tissue, or fluids, such as lungs, heart, blood, bone, joints, or gastrointestinal tract. S. aureus infection, for example, may be associated with sepsis, pneumonia, endocarditis, osteomyelitis, skin infections, food poisoning, toxic shock syndrome, or septic arthritis. S. aureus infections are caused by different strains including MSSA, MRSA, vancomycin-intermediate S. aureus (VISA), and vancomycin-resistant S. aureus (VRSA). S. aureus associated problems with infection are best known for MRSA, but any S. aureus infections can be dangerous and lethal. E. faecium and E. faecalis, are opportunistic pathogens. These bacteria can cause a variety of health problems including urinary tract, intra-abdominal, pelvic, and soft tissue infections, bacteremia, endocarditis, and several uncommon infections such as meningitis, septic arthritis, and pneumonia.

S. aureus along with other staphylococci are the most common causes of persistent biofilm-associated infections. These infections are inherently resistant to existing antibiotics and the host's immune system. S. epdermidis is an opportunistic pathogen, which causes the most biofilm-associated infections on indwelling medical devises and is the most frequent cause of nosocomial sepsis. Two percent of the population carry its drug-resistant variant, methicillin-resistant S. aureus (MRSA), and this bacterium causes the highest number of invasive infections among all antibiotic-resistant bacteria. The failure of antibiotic therapy against S. aureus is due to the development of multidrug-resistant strains and its ability to adopt a persistent non-growing lifestyle and forming biofilms. Both of these features are associated with antibiotic resistance and persistent infection. Similarly, enterococci bacteria that are found in the human intestines and in the female genital tract can cause serious infections. These bacteria are constantly finding new ways to neutralize the effects of antibiotics and vancomycin-resistant enterococci (VRE) infections are becoming common.

Methods for inhibiting growth or proliferation of or killing a microbe are also provided. In some embodiments, administration of any of the compounds disclosed herein to a subject or contacting a microbe with the compound provides for inhibiting growth or proliferation of or killing the microbe.

As used herein, microbe or microorganism is an organism that may exist in a single-cell form or may refer to a colony of cells. Suitably, the microbe is a bacteria. In some embodiments, the bacteria is a Gram-positive bacteria, e.g., S. aureus, K. faecium, B. subtilis, E. faecalis, or S. epidermidis. In other embodiments, the bacteria is a Gram-negative bacteria.

In some embodiments, the microbe is antimicrobial resistant. An antimicrobial resistant microbe is one that has become resistant to one or more antimicrobial agents that are approved for use in the treatment of a subject. Antimicrobial-resistant microbes are more difficult to treat, requiring higher doses, longer treatment regimens, or alternative medications which may prove more toxic. As demonstrated in the Examples, the presently disclosed compounds demonstrated antimicrobial activity against several antimicrobial-resistant microbes, including, S. aureus BAA-2312 (Sa12), which is methicillin resistant; S. aureus ATCC 33591 (Sa91), which is methicillin resistant, S. aureus ATCC 700699 (Sa99), which is methicillin resistant, oxacillin resistant, and has reduced vancomycin susceptibility; S. aureus ATCC 33592 (Sa92), which is methicillin resistant and gentamicin resistant.

In some embodiments, the microbe is antimicrobial persister. Persisters are in a transient, metabolically inactive state. Microbes in this state make conventional antimicrobials that target essential cellular growth processes ineffective. This results in high clinical failure rates of antimicrobial chemotherapy. As demonstrated in the Examples, the presently disclosed compounds demonstrated antimicrobial activity against persisters, including, S. aureus ATCC 700699 (Sa99). Bacterial biofilms are small bacterial communities held together by an extracellular matrix. The biofilm matrix makes bacteria tolerant to harsh conditions and resistant to antibacterial treatments. Biofilms act as a dangerous reservoir of persisters, which can be a nidus for re-infection.

In some embodiments, the methods described herein are practiced in vim. In other embodiments, the methods described herein are practiced in vitro or ex vivo.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be inhibiting the growth or proliferation of or killing the microbe in the subject or reverse the progression or severity of resultant symptoms associated with the microbe.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Compound Synthesis of HT Series

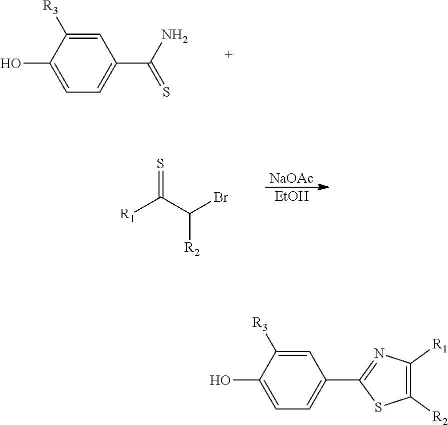

### Scheme 1. HT Series Synthesis of Catechol-Substituted Thiazole Derivatives.

The compounds were synthesized by a procedure previously described. [Alsharif, Z. A.; Alam, M. A., Modular synthesis of thiazoline and thiazole derivatives by using a cascade protocol. RSC Advances 2017, 7 (52), 32647-32651; Alam, A. M., Domino/Cascade and Multicomponent Reactions for the Synthesis of Thiazole Derivatives. Current Organic Chemistry 2022, 26, 1-5]

A mixture of 3,4-dihydroxy thiobenzamide (1 mmol), α-bromo ketone derivative (1.05 mmol), and sodium acetate (1.1 mmol) in 5 mL ethanol was refluxed in a round-bottom flask fitted with a reflux condenser. After the completion of the reaction monitored by TLC, water (˜5 mL) was added to precipitate the product. Filtration followed by further washing with water gave the crude products. Recrystallization with methanol gave the pure compounds.

### Exemplary Compounds for HT Series

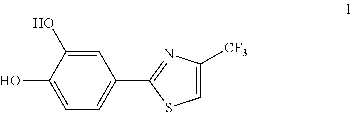 [SMILES: Oc1ccc(-c2nc(C(F)(F)F)cs2)cc1O]

4-(4-(trifluoromethyl)thiazol-2-yl)benzene-1,2-diol (1). ¹H-NMR (400 MHz, DMSO-D6) δ 9.63 (s, 1H), 9.41 (s, 1H), 8.32 (s, 1H), 7.34 (s, 1H), 7.23 (d, J=8.0 Hz, 1H), 6.80 (d, J=8.3 Hz, 1H); ¹³C-NMR (101 MHz, DMSO-D6) δ 171.0, 149.3, 146.4, 143.3, 123.9, 122.5, 119.1, 116.7, 113.8.

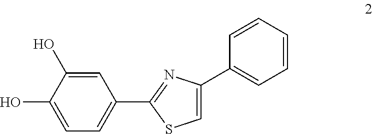 [SMILES: Oc1ccc(-c2nc(-c3ccccc3)cs2)cc1O]

4-(4-phenylthiazol-2-yl)benzene-1,2-diol (2). ¹H-NMR (400 MHz, DMSO-D6) δ 9.46 (br s, 1H), 9.33 (br s, 1H), 7.95 (d, J=8.3 Hz, 3H), 7.40 (d, J=9.9 Hz, 3H), 7.32-7.25 (m, 2H), 6.80 (d, J=8.0 Hz, 1H); ¹³C-NMR (101 MHz, DMSO-D6) δ 168.0, 155.2, 148.5, 146.2, 134.7, 129.3, 128.6, 126.6, 125.2, 118.7, 116.6, 113.8, 113.5.

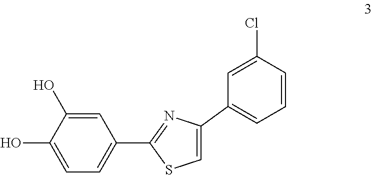 [SMILES: Oc1ccc(-c2nc(-c3cccc(Cl)c3)cs2)cc1O]

### 4-(4-(3-chlorophenyl)thiazol-2-yl)benzene-1,2-diol (3)

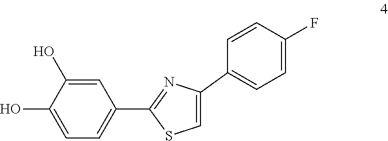 [SMILES: Oc1ccc(-c2nc(-c3ccc(F)cc3)cs2)cc1O]

4-(4-(4-fluorophenyl)thiazol-2-yl)benzene-1,2-diol (4). ¹H-NMR (400 MHz, DMSO-D6) δ 9.47 (s, 1H), 9.32 (s, 1H), 8.00 (t, J=6.1 Hz, 2H), 7.93 (s, 1H), 7.40 (s, 1H), 7.26-7.23 (m, 3H), 6.80 (d, J=8.0 Hz, 1H); ¹³C-NMR (101 MHz, DMSO-D6) δ 168.2, 162.5 (¹⁻²J_{C-F}=243.7 Hz) 154.1, 148.5, 146.2, 131.3, 128.6 (¹⁻¹J_{C-F}=7.1 Hz) 125.1, 118.7, 116.6, 116.2 (¹⁻³J_{C-F}+=21.6 Hz), 113.8, 113.3.

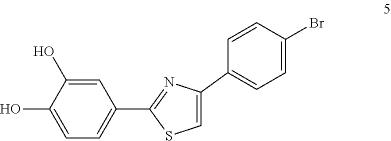 [SMILES: Oc1ccc(-c2nc(-c3ccc(Br)cc3)cs2)cc1O]

4-(4-(4-bromophenyl)thiazol-2-yl)benzene-1,2-diol (5). ¹H-NMR (400 MHz, DMSO-D6) δ 9.47 (s, 1H), 9.32 (s, 1H), 8.03 (s, 1H), 7.91 (d, J=7.3 Hz, 2H), 7.61 (d, J=7.3 Hz, 2H), 7.40 (s, 1H), 7.25 (d, J=8.3 Hz, 1H), 6.79 (d, J=8.1 Hz, 1H); ¹³C-NMR (101 MHz, DMSO-D6) δ 168.3, 153.9, 148.6, 146.2, 133.9, 132.2, 128.6, 125.0, 121.7, 118.7, 116.6, 114.3, 113.8.

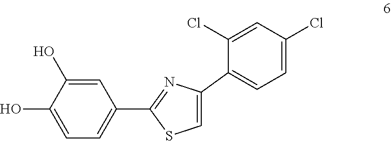 [SMILES: Oc1ccc(-c2nc(-c3ccc(Cl)cc3Cl)cs2)cc1O]

### 4-(4-(2,4-dichlorophenyl)thiazol-2-yl)benzene-1,2-diol (6)

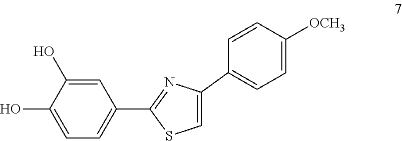 [SMILES: COc1ccc(-c2csc(-c3ccc(O)c(O)c3)n2)cc1]

4-(4-(4-methoxyphenyl)thiazol-2-yl)benzene-1,2-diol (7). ¹H-NMR (400 MHz, DMSO-D6) δ 9.43 (s, 1H), 9.30 (s, 1H), 7.88 (d, J=6.4 Hz, 1H), 7.76 (s, 1H), 7.39 (s, 1H), 7.23 (d, J=7.1 Hz, 1H), 6.96 (d, J=6.4 Hz, 2H), 6.79 (d, J=7.3 Hz, 1H), 3.74 (s, 3H), ¹³C-NMR (101 MHz, DMSO-D6) δ 167.8, 159.7, 155.1, 148.4, 1462, 127.9, 127.6, 125.3, 118.6, 116.5, 114.6, 113.7, 111.4, 55.7.

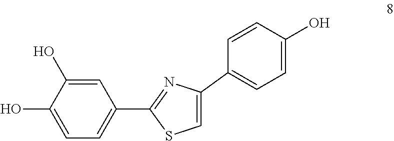 [SMILES: Oc1ccc(-c2csc(-c3ccc(O)c(O)c3)n2)cc1]

4-(4-(4-hydroxyphenyl)thiazol-2-yl)benzene-1,2-diol (8). ¹H-NMR (400 MHz, DMSO-D6) δ 9.44 (s, 1H), 9.31 (s, 1H), 9.18 (s, 1H), 7.74 (s, 2H), 7.55 (d, 0.1=4.1 Hz, 1H), 7.38 (s, 1H), 7.20 (s, 1H), 6.77 (d, J=5.5 Hz, 3H), 3.27 (d, J=4.1 Hz, 9H), 2.45 (s, 2H); ¹³C-NMR (101 MHz, DMSO-D6) δ 167.6, 157.9, 155.6, 148.2, 146.1, 127.9, 126.1, 125.5, 118.5, 116.4, 115.9, 113.7, 110.2.

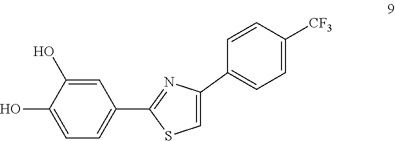 [SMILES: Oc1ccc(-c2nc(-c3ccc(C(F)(F)F)cc3)cs2)cc1O]

4-(4-(4-(trifluoromethyl)phenyl)thiazol-2-yl)benzene-1,2-diol (9). ¹H-NMR (400 MHz, DMSO-D6) δ 9.51 (s, 1H), 9.36 (s, 1H), 8.17 (d, J=8.0 Hz, 3H), 7.78 (d, J=7.8 Hz, 2H), 7.43 (s, 1H), 7.27 (d, J=8.0 Hz, 1H), 6.81 (d, J=8.3 Hz, 1H); ¹³C-NMR (101 MHz, DMSO-D6) δ 168.6, 153.5, 148.7, 146.3, 138.4, 128.5 (¹⁻³J_{C-F}=31.6 Hz), 127.1, 126.3, 126.3, 126.2, 125.0, 123.5, 118.8, 116.6, 116.1, 113.8.

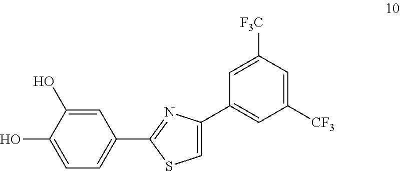 [SMILES: Oc1ccc(-c2nc(-c3cc(C(F)(F)F)cc(C(F)(F)F)c3)cs2)cc1O]

4-(4-(3,5-bis(trifluoromethyl)phenyl)thiazol-2-yl)benzene-1,2-diol (10). ¹H-NMR (400 MHz, DMSO-D6) δ 9.51 (s, 1H), 9.38 (s, 1H), 8.60 (s, 2H), 8.49 (s, 1H), 8.02 (s, 1H), 7.45 (s, 1H), 7.29 (d, J=8.3 Hz, 1H), 6.82 (d, J=8.0 Hz, 1H); ¹³C-NMR (101 MHz, DMSO-D6) δ 168.9, 151.7, 148.9, 146.3, 137.0, 131.6 (¹⁻³J_{C-F}=32 Hz), 126.8, 125.2, 124.7, 122.5, 121.7, 121.6, 119.0, 117.3, 116.6, 113.7, 40.6, 40.4, 40.2, 40.0, 39.8, 39.6, 39.4, 27.1

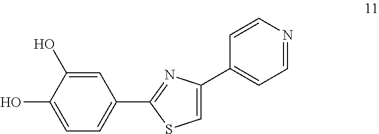 [SMILES: Oc1ccc(-c2nc(-c3ccncc3)cs2)cc1O]

4-(4-(pyridin-4-yl)thiazol-2-yl)benzene-1,2-diol (11). ¹H-NMR (400 MHz, DMSO-D6) δ 9.39 (s, 1H), 8.97 (d, J=7.6 Hz, 1H), 8.82 (d, J=4.4 Hz, 1H), 8.46 (s, 1H), 8.04 (t, J=6.2 Hz, 1H), 7.42 (s, 1H), 7.31 (d, J=7.8 Hz, 1H), 6.83 (d, J=8.0 Hz, 1H); ¹³C-NMR (101 MHz, DMSO-D6) S 169.5, 149.3, 149.0, 146.3, 142.5, 141.4, 141.0, 133.1, 127.5, 124.5, 119.0, 118.5, 116.6, 114.0, 40.7.

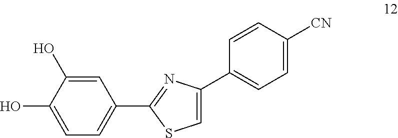 [SMILES: N#Cc1ccc(-c2csc(-c3ccc(O)c(O)c3)n2)cc1]

4-(2-(3,4-dihydroxyphenyl)thiazol-4-yl)benzonitrile (12). ¹H-NMR (400 MHz, DMSO-D6) δ 9.52 (s, 1H), 9.33 (s, 1H), 8.25 (s, 1H), 8.15 (d, J=7.8 Hz, 2H), 7.89 (d, J=7.8 Hz, 2H), 7.42 (s, 1H), 7.27 (d, J=8.3 Hz, 1H), 6.81 (d, J=8.3 Hz, 1H). ¹³C-NMR (101 MHz, DMSO-D6) 6168.7, 153.3, 148.7, 146.3, 138.8, 133.4, 127.2, 124.9, 119.4, 118.8, 117.0, 116.6, 113.8, 110.7.

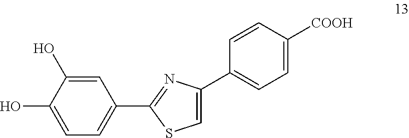 [SMILES: O=C(O)c1ccc(-c2csc(-c3ccc(O)c(O)c3)n2)cc1]

4-(2-(3,4-dihydroxyphenyl)thiazol-4-yl)benzoic acid (13). ¹H-NMR (400 MHz, DMSO-D6) δ 9.8 (br s, 1H), 9.36 (br s, 1H), 8.13 (s, 1H), 8.08 (d, J=7.6 Hz, 2H), 7.98 (d, J=7.6 Hz, 2H), 7.43 (s, 1H), 7.27 (d, J=7.8 Hz, 1H), 6.81 (d, J=7.8 Hz, 1H), ¹³C NMR (101 MHz, DMSO-D6) S 168.4, 167.6, 154.1, 148.6, 146.3, 138.6, 130.4, 126.5, 125.0, 118.8, 116.6, 115.8, 113.8.

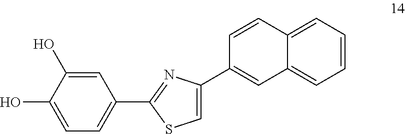 [SMILES: Oc1ccc(-c2nc(-c3ccc4ccccc4c3)cs2)cc1O]

4-(4-(naphthalen-2-yl)thiazol-2-yl)benzene-1,2-diol (14). ¹H-NMR (400 MHz, DMSO-D6) δ 9.43 (d, J=38.1 Hz, 2H), 8.53 (s, 1H), 8.09 (s, 2H), 7.95-7.87 (m, 3H), 7.48 (s, 3H), 7.31 (d, J=7.6 Hz, 1H), 6.83 (d, J=8.0 Hz, 1H); ¹³C-NMR (101 MHz, DMSO-D6) δ 168.2, 155.1, 148.6, 146.3, 133.7, 133.2, 128.9, 128.7, 128.2, 127.1, 126.8, 125.2, 124.8, 118.8, 116.6, 114.1, 113.8.

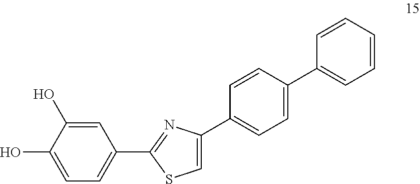 [SMILES: Oc1ccc(-c2nc(-c3ccc(-c4ccccc4)cc3)cs2)cc1O]

4-(4-([1,1′-biphenyl]-4-yl)thiazol-2-yl)benzene-1,2-diol (15). ¹H-NMR (400 MHz, DMSO-D6) δ 9.45 (s, 1H), 8.05 (d, J=7.6 Hz, 2H), 7.98 (s, 1H), 7.78-7.66 (m, 4H), 7.46-7.40 (m, 3H), 7.34-7.28 (m, 2H), 6.83 (d, J=8.0 Hz, 1H); ¹³C-NMR (101 MHz, DMSO-D6) δ 168.1, 154.8, 148.5, 146.3, 140.1, 133.8, 129.5, 128.1, 127.5, 127.1, 127.1, 125.3, 118.7, 116.6, 113.8, 113.6.

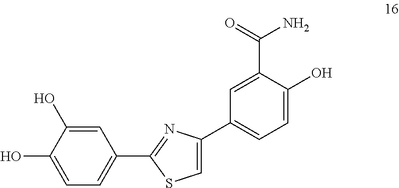 [SMILES: NC(=O)c1cc(-c2csc(-c3ccc(O)c(O)c3)n2)ccc1O]

### 5-(2-(3,4-dihydroxyphenyl)thiazol-4-yl)-2-hydroxybenzamide (16)

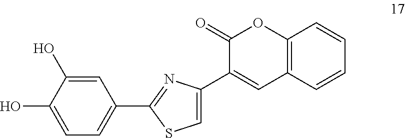 [SMILES: O=c1oc2ccccc2cc1-c1csc(-c2ccc(O)c(O)c2)n1]

3-(2-(3,4-dihydroxyphenyl)thiazol-4-yl)-2H-chromen-2-one (17). ¹H-NMR (400 MHz, DMSO-D6) δ 9.53 (d, J=19.9 Hz, 1H), 8.83 (s, 1H), 8.26 (s, 1H), 7.87 (d, J=7.6 Hz, 1H), 7.59 (t, J=7.6 Hz, 1H), 7.43-7.30 (m, 4H), 6.82 (d, J=8.0 Hz, 1H); ¹³C-NMR (101 MHz, DMSO-D6) δ 167.7, 159.4, 153.1, 148.8, 148.3, 146.3, 140.0, 132.5, 129.6, 125.3, 124.8, 120.8, 119.7, 119.2, 119.0, 116.5, 116.4, 114.0.

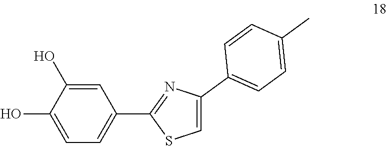 [SMILES: Cc1ccc(-c2csc(-c3ccc(O)c(O)c3)n2)cc1]

### 4-(4-(p-tolyl)thiazol-2-yl)benzene-1,2-diol (18)

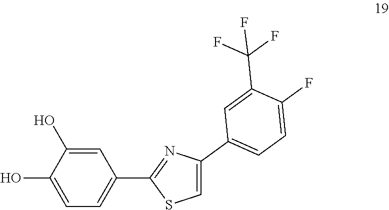 [SMILES: Oc1ccc(-c2nc(-c3ccc(F)c(C(F)(F)F)c3)cs2)cc1O]

### 4-(4-(4-fluoro-3-(trifluoromethyl)phenyl)thiazol-2-yl)benzene-1,2-diol (

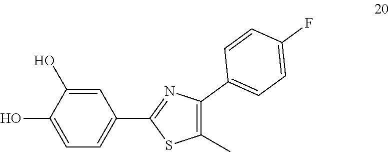 [SMILES: Cc1sc(-c2ccc(O)c(O)c2)nc1-c1ccc(F)cc1]

### 4-(4-(4-fluorophenyl)-5-methylthiazol-2-yl)benzene-1,2-diol (20)

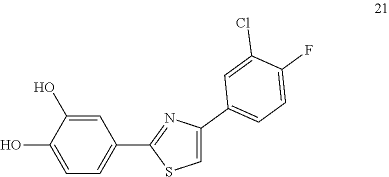 [SMILES: Oc1ccc(-c2nc(-c3ccc(F)c(Cl)c3)cs2)cc1O]

### 4-(4-(3-chloro-4-fluorophenyl)thiazol-2-yl)benzene-1,2-diol (21)

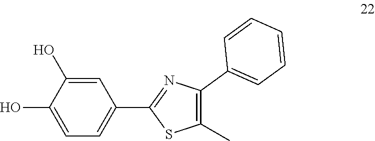 [SMILES: Cc1sc(-c2ccc(O)c(O)c2)nc1-c1ccccc1]

### 4-(5-methyl-4-phenylthiazol-2-yl)benzene-1,2-diol (22)

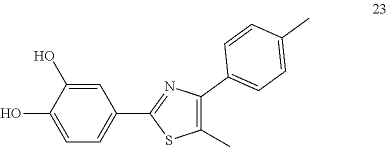 [SMILES: Cc1ccc(-c2nc(-c3ccc(O)c(O)c3)sc2C)cc1]

### 4-(5-methyl-4-(p-tolyl)thiazol-2-yl)benzene-1,2-diol (23)

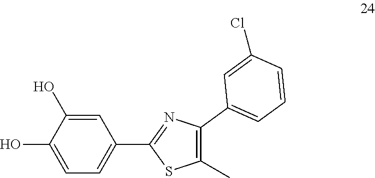 [SMILES: Cc1sc(-c2ccc(O)c(O)c2)nc1-c1cccc(Cl)c1]

### 4-(4-(3-chlorophenyl)-5)benzene-1,2-diol (24)

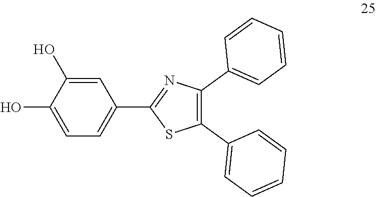 [SMILES: Oc1ccc(-c2nc(-c3ccccc3)c(-c3ccccc3)s2)cc1O]

### 4-(4,5-diphenylthiazol-2-yl)benzene-1,2-diol (25)

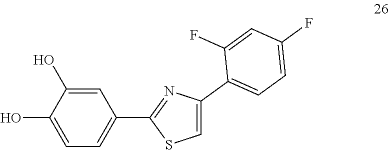 [SMILES: Oc1ccc(-c2nc(-c3ccc(F)cc3F)cs2)cc1O]

### 4-(4-(2,4-difluorophenyl)thiazol-2-yl)benzene-1,2-diol (26)

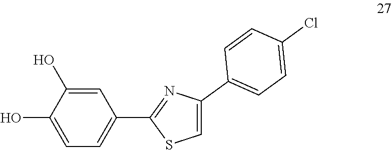 [SMILES: Oc1ccc(-c2nc(-c3ccc(Cl)cc3)cs2)cc1O]

### 4-(4-(4-chlorophenyl)thiazol-2-yl)benzene-1,2-diol (27)

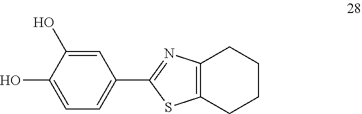 [SMILES: Oc1ccc(-c2nc3c(s2)CCCC3)cc1O]

### 4-(4,5,6,7-tetrahydrobenzo[d]thiazol-2-yl)benzene-1,2-diol (28)

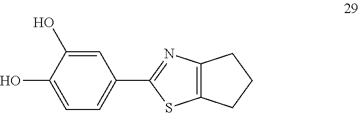 [SMILES: Oc1ccc(-c2nc3c(s2)CCC3)cc1O]

### 4-(5,6-dihydro-4H-cyclopenta[d]thiazol-2-yl)benzene-1,2-diol (29)

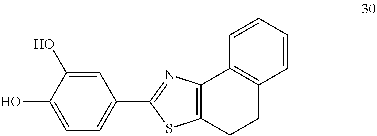 [SMILES: Oc1ccc(-c2nc3c(s2)CCc2ccccc2-3)cc1O]

### 4-(4,5-dihydronaphtho[1,2-d]thiazol-2-yl)benzene-1,2-diol (30)

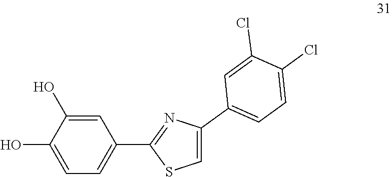 [SMILES: Oc1ccc(-c2nc(-c3ccc(Cl)c(Cl)c3)cs2)cc1O]

### 4-(4-(3,4-dichlorophenyl)thiazol-2-yl)benzene-1,2-diol (31)

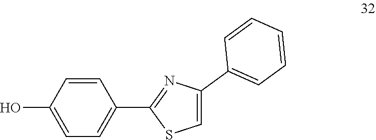 [SMILES: Oc1ccc(-c2nc(-c3ccccc3)cs2)cc1]

### 4-(4-phenylthiazol-2-yl)phenol (32)

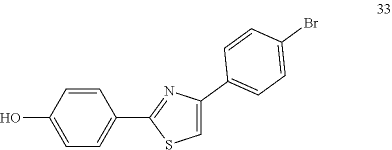 [SMILES: Oc1ccc(-c2nc(-c3ccc(Br)cc3)cs2)cc1]

### 4-(4-(4-bromophenyl)thiazol-2-yl)phenol (33)

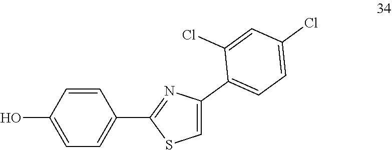 [SMILES: Oc1ccc(-c2nc(-c3ccc(Cl)cc3Cl)cs2)cc1]

### 4-(4-(2,4-dichlorophenyl)thiazol-2-yl)phenol (34)

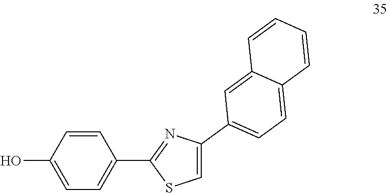 [SMILES: Oc1ccc(-c2nc(-c3ccc4ccccc4c3)cs2)cc1]

### 4-(4-(naphthalen-2-yl)thiazol-2-yl)phenol (35)

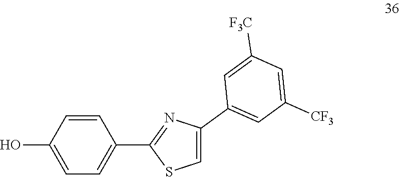 [SMILES: Oc1ccc(-c2nc(-c3cc(C(F)(F)F)cc(C(F)(F)F)c3)cs2)cc1]

### 4-(4-(3,5-bis(trifluoromethyl)phenyl)thiazol-2-yl)phenol (36)

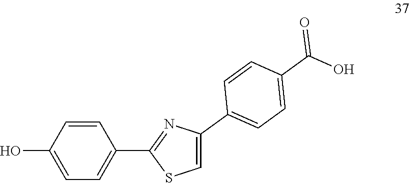 [SMILES: O=C(O)c1ccc(-c2csc(-c3ccc(O)cc3)n2)cc1]

### 4-(2-(4-hydroxyphenyl)thiazol-4-yl)benzoic acid (37)

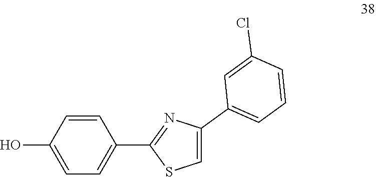 [SMILES: Oc1ccc(-c2nc(-c3cccc(Cl)c3)cs2)cc1]

### 4-(4-(3-chlorophenyl)thiazol-2-yl)phenol (38)

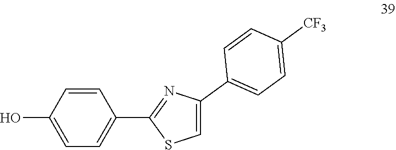 [SMILES: Oc1ccc(-c2nc(-c3ccc(C(F)(F)F)cc3)cs2)cc1]

### 4-(4-(4(trifluoromethyl)phenyl)thiazol-2-yl)phenol (39)

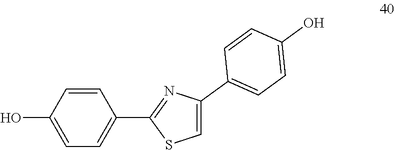 [SMILES: Oc1ccc(-c2csc(-c3ccc(O)cc3)n2)cc1]

### 4,4′-(thiazole-2,4-diyl)diphenol (40)

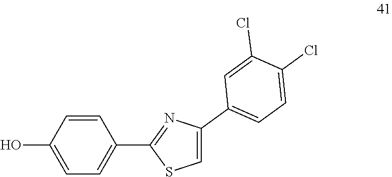 [SMILES: Oc1ccc(-c2nc(-c3ccc(Cl)c(Cl)c3)cs2)cc1]

### 4-(4-(3,4-dichlorophenyl)thiazol-2-yl)phenol (41)

### Experimental Procedure for Minimum Inhibitory Concentration (MIC)

The broth microdilution method was utilized to determine MIC values of novel thiazole derivatives against different clinically important Gram-positive bacteria according to the guidelines outlined by the Clinical and Laboratory Standards Institute (CLSI) as reported in our recent papers.¹⁻⁵ The starting concentration of compounds for MIC determination was 64 μg/mL serially diluted down the wells and the MIC values were recorded in duplicates in three independent experiments on different days.

TABLE 1
HT Series: Minimum inhibitory concentration (MIC) for S. aureus ATCC 700699 (Sa 99), S. aureus
ATCC 33592 (Sa 92), S. aureus ATCC 33591 (Sa 91), S. aureus ATCC 25923 (Sa 23), S. aureus
ATCC BAA-2312 (Sa 12), E. faecalis ATCC 29212 (Efs 12), Sa UAMS-1 MRSA ATCC 49230(Sa UAMS),
S. aureus subsp. aureus Rosenbach ATCC 25904 (SA Newman), S. aureus LAC MRSA (Sa LAC), S. aureus
subsp. aureus Rosenbach BAA-1717 (Sa USA 300), E. faecalis ATCC 51299 (Efs 99), E. faecium
ATCC 700221 (Efm 21), S. epidermidis ATCC 700296 (Se), and B. subtilis ATCC 6633 (Bs)..
Individual replicates collected on different days are delineated by a comma. Units are μg/mL.

| | | | | | | | | | | SA | | | | |
|---|---|---|---|---|---|---|---|---|---|---|---|---|---|---|
| | | Sa | Sa | | Sa | | Sa | Sa | Sa | USA | | Efs | Efm | |
| | Sa 99 | 92 | 91 | Sa 23 | 12 | Efs 12 | UAMS | Newman | LAC | 300 | Se | 99 | 21 | Bs |
| 1 | >32, >32 | — | — | >32, >32 | — | >32, >32 | — | — | — | — | — | — | — | — |
| 2 | 32, 16, 16 | 16 | 16 | 8, 16, 16 | 32 | >32, >32, >32 | 32 | 32 | 32 | — | — | — | — | — |
| 3 | 4, 4, 4 | 8 | 4 | 2, 2, 2 | 8 | 16, 8, 16 | 8 | 4 | 8 | — | — | — | — | — |
| 4 | 16, 4, 16 | 16 | 8 | 4, 8, 8 | 16 | 32, 32, 32 | 16 | 8 | 16 | — | — | — | — | — |
| 5 | 2, 2, 2 | 8 | 4 | 2, 2, 2 | 8 | 16, 2, 16 | 8 | 4 | 4 | 8 | 8 | 16 | 16 | 16 |
| 6 | 2, 2, 2 | 8 | 4 | 2, 2, 2 | 8 | 16, 2, 8 | 8 | 4 | 8 | — | — | — | — | — |
| 7 | 32, 32 | — | — | 16, 16 | — | >32, >32 | — | — | — | — | — | — | — | — |
| 8 | 16, 8, 8 | 32 | 32 | 16, 16, 16 | 16 | >32, >32, >32 | 32 | 16 | 16 | — | — | — | — | — |
| 9 | 2, 2, 2 | 8 | 4 | 2, 2, 2 | 4 | 16, 2, 8 | 4 | 4 | 8 | 4 | 8 | 16 | 16 | 8 |
| 10 | 2, 2, 2 | 2 | 2 | 2, 1, 2 | 2 | 16, 2, 8 | 2 | 2 | 2 | 4 | 8 | 8 | 8 | 8 |
| 11 | >32, >32 | — | — | >32, >32 | — | >32, >32 | — | — | — | — | — | — | — | — |
| 12 | >32, >32 | — | — | >32, >32 | — | >32, >32 | — | — | — | — | — | — | — | — |
| 13 | >32, >32 | — | — | >32, >32 | — | >32, >32 | — | — | — | — | — | — | — | — |
| 14 | 2, 2, 2 | 8 | 8 | 2, 2, 2 | 8 | 16, 16, 16 | 8 | 8 | 4 | — | — | — | — | — |
| 15 | >32, >32 | — | — | >32, >32 | — | >32, >32 | — | — | — | — | — | — | — | — |
| 16 | >32, >32 | — | — | >32, >32 | — | >32, >32 | — | — | — | — | — | — | — | — |
| 17 | 16, 32 | — | — | >32, >32 | — | >32, >32 | — | — | — | — | — | — | — | — |
| 21 | 8 | 8 | 4 | 8 | 8 | 16 | 4 | 4 | — | 4 | 8 | 16 | 16 | 16 |
| 31 | 4 | 4 | 4 | 4 | 4 | 16 | 4 | 4 | — | 4 | 4 | 16 | 16 | 16 |
| 34 | 4 | 4 | 4 | 4 | 4 | 8 | 4 | 4 | — | 4 | 4 | 8 | 8 | 4 |
| 36 | 2 | 2 | 2 | 2 | 2 | 2 | 2 | 2 | — | 2 | 2 | 2 | 2 | 2 |
| 38 | 4 | 4 | 4 | 4 | 4 | 8 | 4 | 4 | — | 4 | 4 | 8 | 8 | 4 |
| 39 | 4 | 4 | 4 | 2 | 2 | >32 | 4 | 4 | — | 4 | 2 | >32 | 32 | 4 |
| 41 | 1 | 1 | 1 | 1 | 1 | 2 | 2 | 2 | — | 1 | 1 | 2 | 2 | 4 |
| Vanc. | 2 | 1 | 2 | 1 | 1 | 2 | 2 | 2 | 1 | — | — | — | — | — |
| Dapt. | — | — | — | — | — | — | — | — | — | — | — | 1 | 4 | — |

Vanc.: Vancomycin
Dapt.: Daptomycin

### Compound Synthesis of HTM Series

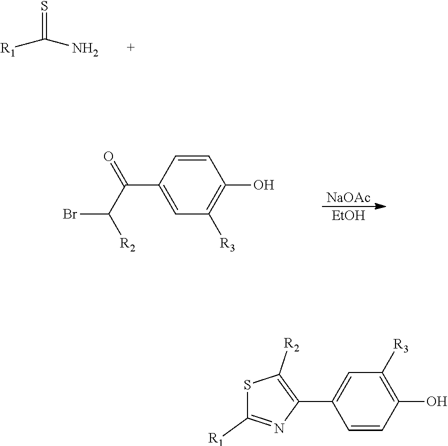

### Scheme 2. HTM Series Synthesis of Catechol-Substituted Thiazole Derivatives.

The compounds were synthesized by by same procedure as described above for the HT series compounds with altered starting material. A mixture of reactants and sodium acetate in ethanol was refluxed in a round-bottom flask fitted with a reflux condenser. After the completion of the reaction monitored by TLC, water (˜5 mL) was added to precipitate the product. Filtration followed by further washing with water gave the crude products. Recrystallization with methanol gave the pure compounds.

### Exemplary Compounds for HTM Series

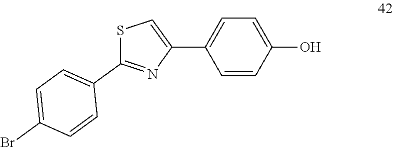 [SMILES: Oc1ccc(-c2csc(-c3ccc(Br)cc3)n2)cc1]

4-(2-(4-bromophenyl)thiazol-4-yl)phenol (42). 1H-NMR (400 MHz, DMSO-D6) δ 9.66 (s, 1H), 7.93-7.90 (m, 3H), 7.82 (d, J=8.5 Hz, 2H), 7.68 (dd, J=8.5, 2.8 Hz, 2H), 6.81 (d, J=8.5 Hz, 2H); 13C-NMR (101 MHz, DMSO-D6) δ 165.8, 158.2, 156.2, 132.8, 132.7, 128.5, 128.1, 125.7, 124.0, 116.1, 112.8.

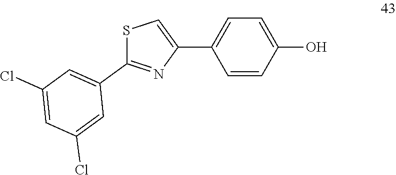 [SMILES: Oc1ccc(-c2csc(-c3cc(Cl)cc(Cl)c3)n2)cc1]

4-(2-(3,5-dichlorophenyl)thiazol-4-yl)phenol (43). ¹H-NMR (400 MHz, DMSO-D6) δ 9.69 (s, 1H), 7.99 (d, J=11.2 Hz, 3H), 7.84 (d, J=8.3 Hz, 2H), 7.72 (s, 1H), 6.81 (d, J=8.3 Hz, 2H), 3.36 (s, 41H), 2.46 (s, 3H); ¹³C-NMR (101 MHz, DMSO-D6) δ 163.7, 158.3, 156.4, 136.7, 135.6, 129.9, 128.2, 125.4, 125.0, 116.1, 114.1.

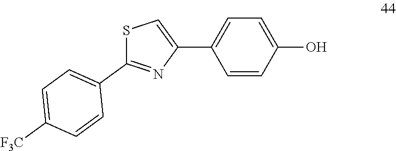 [SMILES: Oc1ccc(-c2csc(-c3ccc(C(F)(F)F)cc3)n2)cc1]

4-(2-(4-(trifluoromethyl)phenyl)thiazol-4-yl)phenol (44). ¹H-NMR (400 MHz, DMSO-D6) δ 9.70 (d, J=5.7 Hz, 1H), 8.17 (t, J=6.2 Hz, 2H), 7.99 (d, J=6.0 Hz, 1H), 7.83 (d, J=6.4 Hz, 4H), 6.84-6.80 (m, 2H).

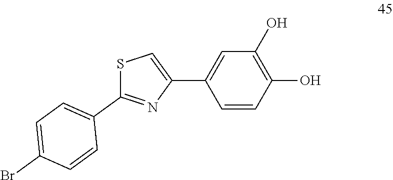 [SMILES: Oc1ccc(-c2csc(-c3ccc(Br)cc3)n2)cc1O]

### 2-(4-bromophenylthiazol-4-yl)benzene-1,2-diol (45)

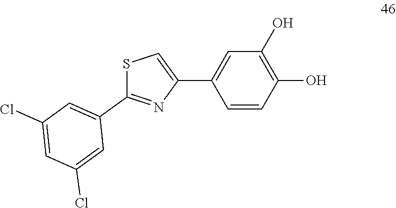 [SMILES: Oc1ccc(-c2csc(-c3cc(Cl)cc(Cl)c3)n2)cc1O]

### 4-(2-(3,5-dichlorophenyl)thiazol-4-yl)benzene-1,2-diol (46)

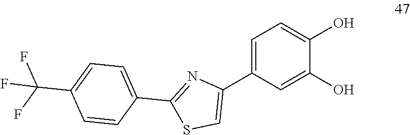 [SMILES: Oc1ccc(-c2csc(-c3ccc(C(F)(F)F)cc3)n2)cc1O]

4-{2-[4-(trifluoromethyl)phenyl]-1,3-thiazol-4-yl}benzene-1,2-diol (47). 1H-NMR (400 MHz, DMSO-D6) δ 9.15 (s, 1H), 8.16, (t, J=7.3 Hz, 2H), 7.93 (d, J=7.9 Hz, 1H), 7.87-7.83 (m, 2H), 7.44-7.27 (m, 2H), 6.78 (t, J=8.0 Hz, 1H).

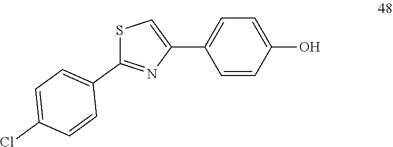 [SMILES: Oc1ccc(-c2csc(-c3ccc(Cl)cc3)n2)cc1]

### 4-(2-(4-chlorophenyl)thiazol-4-yl)phenol (48)

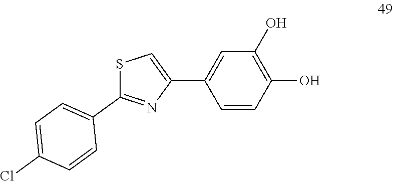 [SMILES: Oc1ccc(-c2csc(-c3ccc(Cl)cc3)n2)cc1O]

4-(2-(4-chlorophenyl)thiazol-4-yl)benzene-1,2-diol (49). ¹³C-NMR (101 MHz, DMSO-D6) δ 165.5, 156.4, 146.4, 145.9, 135.2, 132.5, 129.8, 128.3, 126.1, 118.2, 116.3, 114.3, 112.8.

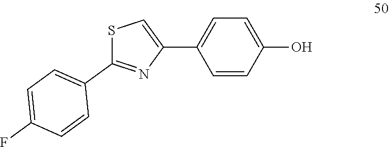 [SMILES: Oc1ccc(-c2csc(-c3ccc(F)cc3)n2)cc1]

4-(2-(4-fluorophenyl)thiazol-4-yl)phenol (50). ¹H-NMR (400 MHz, DMSO-D6) δ 9.66 (d, J=6.4 Hz, 1H), 8.05-7.99 (m, 2H), 7.88-7.80 (m, 3H), 7.36-7.29 (m, 2H), 6.83-6.79 (m, 2H) peak splitting is not good; ¹³C-NMR (101 MHz, DMSO-D6) δ 165.9, 163.7 (¹J_{C-F}=248.0 Hz), 158.2, 156.0, 130.3, 128.9 (³J_{C-F}=8.6 Hz), 128.1, 125.8, 116.7 (¹J_{C-F}=22.1 Hz), 116.0, 112.4.

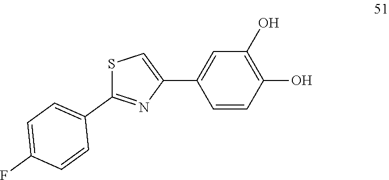 [SMILES: Oc1ccc(-c2csc(-c3ccc(F)cc3)n2)cc1O]

4-(2-(4-fluorophenyl)thiazol-4-yl)benzene-1,2-diol (51). ¹H-NMR (400 MHz, DMSO-D6) δ 9.18 (s, 1H), 9.08 (s, 1H), 7.99 (s, 2H), 7.79 (s, 1H), 7.42 (s, 1H), 7.34-7.27 (m, 3H), 6.78 (d, J=7.5 Hz, 1H); ¹³C-NMR (101 MHz, DMSO-D6) δ 165.7, 163.6 (¹J_{C-F}=247.5 Hz), 156.2, 146.3, 145.9, 130.4, 128.8 (³J_{C-F}=8.6 Hz), 126.2, 118.1, 116.8 (²J_{C-F}=22.1 Hz), 116.3, 114.3, 112.4.

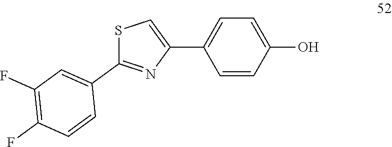 [SMILES: Oc1ccc(-c2csc(-c3ccc(F)c(F)c3)n2)cc1]

4-(2-(3,4-difluorophenyl)thiazol-4-yl)phenol (52). ¹H-NMR (400 MHz, DMSO-D6) δ 9.68 (d, J=4.9 Hz, 1H), 8.02-7.98 (m, 1H), 7.89 (d, J=5.0 Hz, 1H), 7.83 (d, J=6.4 Hz, 2H), 7.56-7.52 (m, 1H), 6.82 (d, J=6.2 Hz, 2H); ¹³C-NMR (101 MHz, DMSO-D6) δ 164.6, 158.2, 156.2, 131.2, 131.2, 128.2, 128.2, 125.6, 123.8, 123.7, 119.1, 118.9, 116.0, 115.6, 115.5, 113.1.

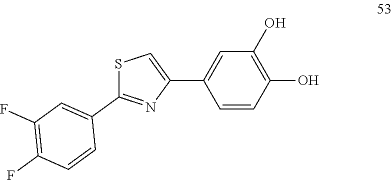 [SMILES: Oc1ccc(-c2csc(-c3ccc(F)c(F)c3)n2)cc1O]

4-(2-(3,4-difluorophenyl)thiazol-4-yl)benzene-1,2-diol (53). ¹H-NMR (400 MHz, DMSO-D6) δ 9.18 (s, 1H), 9.06 (s, 1H), 8.00 (d, J=7.1 Hz, 1H), 7.82 (d, J=19.7 Hz, 2H), 7.59-7.54 (m, 1H), 7.41 (s, 1H), 7.28 (d, J=7.1 Hz, 1H), 6.77 (d, J=7.5 Hz, 1H).

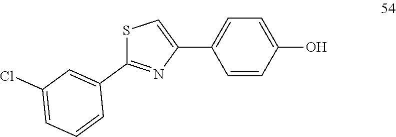 [SMILES: Oc1ccc(-c2csc(-c3cccc(Cl)c3)n2)cc1]

4-(2-(3-chlorophenyl)thiazol-4-yl)phenol (54). ¹H-NMR (400 MHz, DMSO-D6) δ 9.67 (s, 0H), 8.01 (s, 0H), 7.95 (s, 0H), 7.91 (d, J=4.8 Hz, 0H), 7.84 (d, J=7.5 Hz, 1H), 7.52 (s, 1H), 6.81 (d, J=7.6 Hz, 1H); ¹³C-NMR (101 MHz, DMSO-D6) δ 165.3, 158.3, 156.3, 135.6, 134.5, 131.7, 130.4, 128.2, 125.9, 125.7, 125.4, 116.1, 113.1.

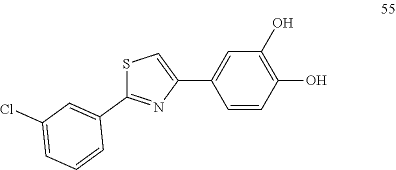 [SMILES: Oc1ccc(-c2csc(-c3cccc(Cl)c3)n2)cc1O]

4-(2-(3-chiorophenyl)thiazol-4-yl)benzene-1,2-diol (55). ¹H-NMR (400 MHz, DMSO-D6) δ 9.17 (s, 1H), 9.08 (s, 1H), 7.99 (s, 1H), 7.87 (s, 2H), 7.53 (s, 2H), 7.43 (s, 1H), 7.28 (d, J=7.9 Hz, 1H), 6.77 (d, J=8.0 Hz, 1H).

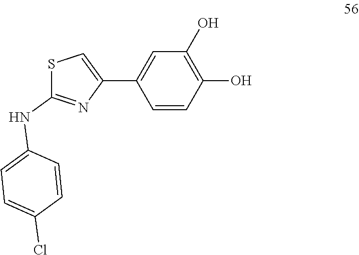 [SMILES: Oc1ccc(-c2csc(Nc3ccc(Cl)cc3)n2)cc1O]

4-(2-((4-chlorophenyl)amino)thiazol-4-yl)benzene-1,2-diol (56). ¹H-NMR (400 MHz, DMSO-D6) δ 10.29 (s, 1H), 9.05 (s, 1H), 8.95 (s, 1H), 7.71 (d, J=7.9 Hz, 2H), 7.33 (d, J=7.7 Hz, 2H), 7.27 (s, 1H), 7.15 (d, J=8.1 Hz, 1H), 6.97 (s, 1H), 6.72 (d, J=8.0 Hz, 1H); ¹³C-NMR (101 MHz, DMSO-D6) δ 162.8, 151.1, 145.8, 145.7, 140.7, 129.3, 126.8, 124.8, 118.7, 117.7, 116.2, 114.0, 100.8.

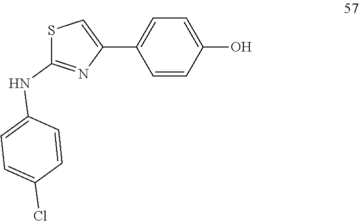 [SMILES: Oc1ccc(-c2csc(Nc3ccc(Cl)cc3)n2)cc1]

4-(2-((4-chlorophenyl)amino)thiazol-4-yl)phenol (57). ¹H-NMR (400 MHz, DMSO-D6) 810.33 (s, 1H), 9.55 (s, 1H), 7.71 (t, J=7.7 Hz, 4H), 7.34 (d, J=8.0 Hz, 2H), 7.05 (s, 1H), 6.77 (d, J=7.7 Hz, 2H); ¹³C-NMR (101 MHz, DMSO-D6) δ 163.0, 157.7, 150.9, 140.7, 129.4, 127.6, 126.3, 124.8, 118.7, 115.9, 100.8.

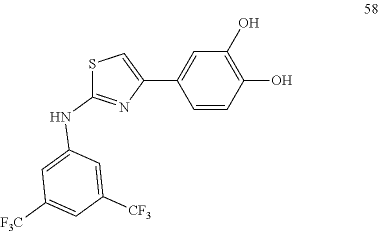 [SMILES: Oc1ccc(-c2csc(Nc3cc(C(F)(F)F)cc(C(F)(F)F)c3)n2)cc1O]

4-(2-((3,5-bis(trifluoromethyl)phenyl)amino)thiazol-4-yl)benzene-1,2-diol (58). ¹H-NMR (400 MHz, DMSO-D6) δ 10.93 (s, 1H), 9.21 (s, 1H), 8.90 (s, 1H), 8.39 (s, 2H), 7.56 (s, 1H), 7.23 (s, 1H), 7.16 (d, J=8.0 Hz, 1H) 7.13 (s, 1H), 6.74 (d, J=8.0 Hz, 1H).

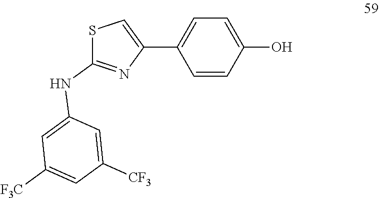 [SMILES: Oc1ccc(-c2csc(Nc3cc(C(F)(F)F)cc(C(F)(F)F)c3)n2)cc1]

4-(2-((3,5-bis(trifluoromethyl)phenyl)amino)thiazol-4-yl)phenol (59). ¹H-NMR (400 J 11 Hz, DMSO-D6) δ 10.96 (s, 1H), 9.62 (s, 1H), 8.41 (s, 2H), 7.68 (d, J=8.4 Hz, 2H), 7.57 (s, 1H), 7.21 (s, 1H), 6.78 (d, J=8.3 Hz, 2H).

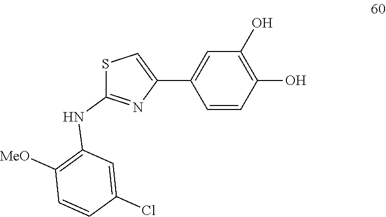 [SMILES: COc1ccc(Cl)cc1Nc1nc(-c2ccc(O)c(O)c2)cs1]

4-(2-((3-methoxyphenyl)amino)thiazol-4-yl)benzene-1,2-diol (60). ¹H-NMR (400 MHz, DMSO-D6) δ 9.73 (d, J=6.0 Hz, 0H), 9.10 (d, J=6.2 Hz, 0H), 8.96 (d, J=6.2 Hz, 0H), 8.71 (d, J=4.8 Hz, 0H), 7.23 (d, J=5.3 Hz, 0H), 7.13 (s, 0H), 6.98-6.93 (m, 1H), 6.73 (d, J=7.1 Hz, 0H), 3.83 (d, J=6.1 Hz, 1H). ¹³C-NMR (101 MHz, DMSO-D6) δ 163.0, 150.6, 146.8, 145.8, 145.7, 132.1, 127.0, 124.8, 120.8, 117.6, 117.1, 116.2, 113.9, 112.4, 102.0, 56.6, 40.6, 40.4, 40.2, 40.0, 39.8, 39.6, 39.4.

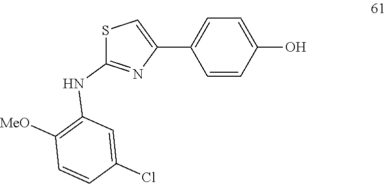 [SMILES: COc1ccc(Cl)cc1Nc1nc(-c2ccc(O)cc2)cs1]

4-(2-((5-chloro-2-methoxyphenyl)amino)thiazol-4-yl)phenol (61). ¹H-NMR (400 MHz, DMSO-D6) δ 9.78 (s, 1H), 9.56 (s, 1H), 8.75 (s, 1H), 7.66 (d, J=8.1 Hz, 2H), 7.07 (s, 1H), 6.99-6.92 (m, 2H), 6.78 (d, J=8.3 Hz, 2H), 3.83 (s, 3H).

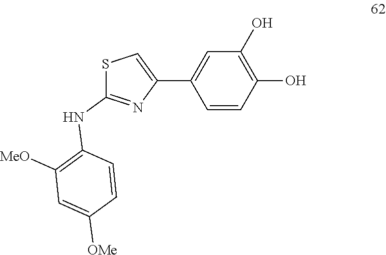 [SMILES: COc1ccc(Nc2nc(-c3ccc(O)c(O)c3)cs2)c(OC)c1]

### 4-(2-((2,4-dimethoxyphenyl)amino)thiazol-4-yl)benzene-1,2-diol (62)

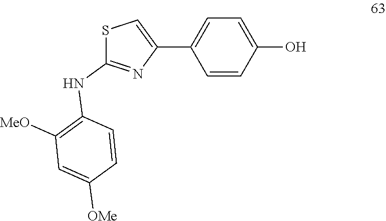 [SMILES: COc1ccc(Nc2nc(-c3ccc(O)cc3)cs2)c(OC)c1]

4-(2-((2,4-dimethoxyphenyl)amino)thiazol-4-yl)phenol (63). ¹³C-NMR (101 MHz, DMSO-D6) δ 165.6, 157.5, 156.2, 151.1, 150.6, 127.5, 126.8, 124.3, 1215, 115.8, 104.8, 100.0, 99.7, 56.3, 55.8.

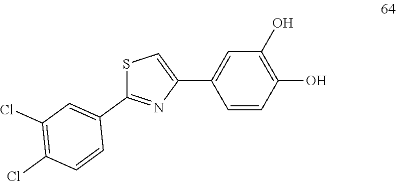 [SMILES: Oc1ccc(-c2csc(-c3ccc(Cl)c(Cl)c3)n2)cc1O]

4-(2-(3,4-dichlorophenyl)thiazol-4-yl)benzene-1,2-diol (64). ¹³C-NMR (101 MHz, DMSO-D6) δ 162.4, 151.1, 145.9, 145.7, 141.7, 131.7, 131.3, 126.7, 122.4, 118.2, 117.7, 117.3, 116.2, 113.9, 101.5.

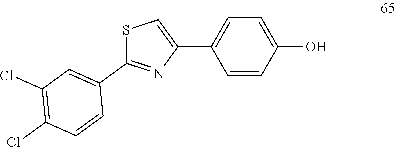 [SMILES: Oc1ccc(-c2csc(-c3ccc(Cl)c(Cl)c3)n2)cc1]

4-(2-(3,4-dichlorophenyl)thiazol-4-yl)phenol (65). ¹H-NMR (400 MHz, DMSO-D6) S 9.69 (s, 1H), 8.17 (s, 1H), 7.95-7.72 (m, 5H), 6.81 (s, 2H). ¹³C-NMR (101 MHz, DMSO-D6) δ 164.2, 158.3, 156.4, 134.1, 133.1, 132.6, 132.0, 128.2, 127.9, 126.7, 125.6, 116.1, 113.5.

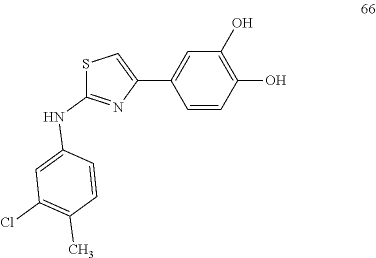 [SMILES: Cc1ccc(Nc2nc(-c3ccc(O)c(O)c3)cs2)cc1Cl]

4-(2-((3-chloro-4-methylphenyl)amino)thiazol-4-yl)benzene-1,2-diol (66). ¹H-NMR (400 MHz, DMSO-D6) δ 10.26 (s, 1H), 9.09 (s, 1H), 8.98 (s, 1H), 7.88 (s, 1H), 7.44 (d, J=6.8 Hz, 1H), 7.25 (s, 2H), 7.14 (d, J=6.8 Hz, 1H), 6.97 (s, 1H), 6.73 (d, J=7.3 Hz, 1H), 2.23 (s, 3H).

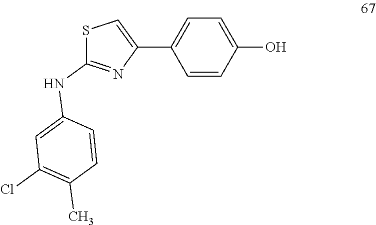 [SMILES: Cc1ccc(Nc2nc(-c3ccc(O)cc3)cs2)cc1Cl]

4-(2-((3-chloro-4-methylphenyl)amino)thiazol-4-yl)phenol (67). ¹¹C-NMR (101 MHz, DMSO-D6) δ 163.0, 157.7, 150.8, 140.9, 133.8, 131.9, 127.8, 127.5, 126.4, 117.1, 116.0, 115.9, 100.8, 19.4.

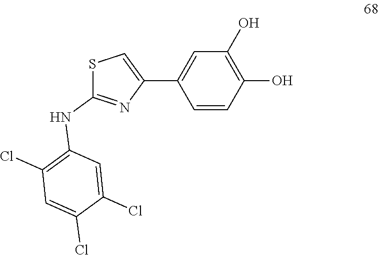 [SMILES: Oc1ccc(-c2csc(Nc3cc(Cl)c(Cl)cc3Cl)n2)cc1O]

4-(2-((2,4,5-trichlorophenyl)amino)thiazol-4-yl)benzene-1,2-diol (68). ¹³C-NMR (101 MHz, DMSO-D6) δ 162.6, 150.6, 146.0, 145.8, 138.2, 130.8, 130.6, 126.7, 123.4, 120.8, 120.5, 117.6, 116.2, 113.9, 103.3.

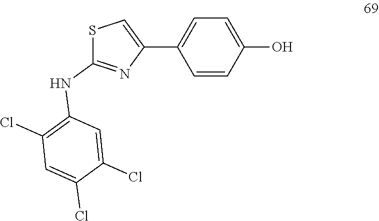 [SMILES: Oc1ccc(-c2csc(Nc3cc(Cl)c(Cl)cc3Cl)n2)cc1]

4-(2-((2,4,5-trichlorophenyl)amino)thiazol-4-yl)phenol (69). 1H-NMR (400 MHz, DMSO-D6) δ 9.97 (d, J=3.2 Hz, 1H), 9.60 (d, J=4.1 Hz, 1H), 9.03 (d, J=4.2 Hz, 1H), 7.81 (d, J=4.1 Hz, 1H), 7.66-7.64 (m, 2H), 7.19 (d, J=3.9 Hz, 1H), 6.79-6.76 (m, 2H).

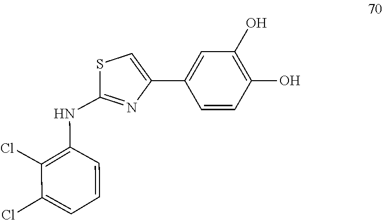 [SMILES: Oc1ccc(-c2csc(Nc3cccc(Cl)c3Cl)n2)cc1O]

### 4-(2-((2,3-dichlorophenyl)amino)thiazol-4-yl)benzene-1,2-diol (70)

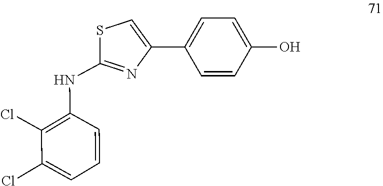 [SMILES: Oc1ccc(-c2csc(Nc3cccc(Cl)c3Cl)n2)cc1]

4-(2-((2,3-dichlorophenyl)amino)thiazol-4-yl)phenol (71). ¹³C-NMR (101 MHz, DMSO-D6) S 163.4, 157.7, 150.5, 140.2, 132.4, 128.8, 127.6, 126.3, 123.7, 120.5, 119.2, 115.9, 102.7.

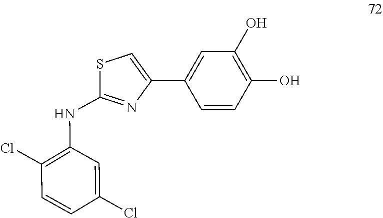 [SMILES: Oc1ccc(-c2csc(Nc3cc(Cl)ccc3Cl)n2)cc1O]

4-(2-((2,5-dichlorophenyl)amino)thiazol-4-yl)benzene-1,2-diol (72). ¹³C-NMR (101 MHz, DMSO-D6) δ 162.8, 150.5, 145.9, 145.8, 139.2, 132.5, 131.3, 126.7, 122.5, 120.1, 119.5, 117.6, 116.2, 113.8, 103.1.

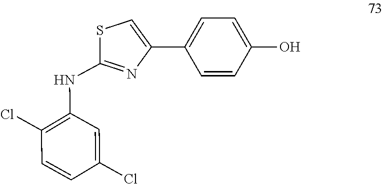 [SMILES: Oc1ccc(-c2csc(Nc3cc(Cl)ccc3Cl)n2)cc1]

4-(2-((2,5-dichlorophenyl)amino)thiazol-4-yl)phenol (73). ¹³C-NMR (101 MHz, DMSO-D6) δ 162.9, 157.7, 150.3, 139.2, 132.5, 131.3, 127.4, 126.2, 122.5, 120.0, 119.5, 116.0, 103.0.

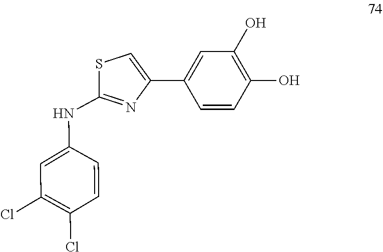 [SMILES: Oc1ccc(-c2csc(Nc3ccc(Cl)c(Cl)c3)n2)cc1O]

### 4-(2-((3,4-dichlorophenyl)amino)thiazol-4-yl)benzene-1,2-diol (74)

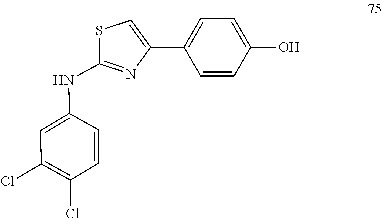 [SMILES: Oc1ccc(-c2csc(Nc3ccc(Cl)c(Cl)c3)n2)cc1]

4-(2-((3,4-dichlorophenyl)amino)thiazol-4-yl)phenol (75). ¹³C-NMR (101 MHz, DMSO-D6) δ 162.6, 157.8, 150.9, 141.7, 131.7, 131.3, 127.6, 126.2, 122.5, 118.2, 117.4, 115.9, 101.5.

TABLE 2
HTM Series: Minimum inhibitory concentration (MIC) for S. aureus ATCC 700699 (Sa 99), S. aureus
ATCC 33592 (Sa 92), S. aureus ATCC 33591 (Sa 91), S. aureus ATCC 25923 (Sa 23), S. aureus
ATCC BAA-2312 (Sa 12), E. faecalis ATCC 29212 (Efs 12), Sa UAMS-1 MRSA ATCC 49230(Sa UAMS),
S. aureus subsp. aureus Rosenbach ATCC 25904 (SA Newman), S. aureus LAC MRSA (Sa LAC),
S. aureus subsp. aureus Rosenbach BAA-1717 (Sa USA 300), E. faecalis ATCC 51299 (Efs 99),
E. faecium ATCC 700221 (Efm 21), S. epidermidis ATCC 700296 (Se), and B. subtilis ATCC
6633 (Bs). Units are μg/mL.

| | Sa UAMS-1 | Sa Newman | Sa USA 300 | Sa 99 | Sa 92 | Sa 91 | Sa 23 | Sa 12 | Se | Efs 99 | Efs 12 | Efm 21 |
|---|---|---|---|---|---|---|---|---|---|---|---|---|
| 43 | 1 | 1 | 1 | 1 | 1 | 1 | 1 | 1 | 1 | 2 | 2 | 2 |
| 47 | 8 | 8 | 8 | 8 | 8 | 8 | 4 | 4 | 8 | 8 | 16 | 8 |
| 58 | 4 | 4 | 4 | 4 | 4 | 4 | 4 | 4 | 4 | 4 | 4 | 4 |
| 59 | 2 | 1 | 1 | 1 | 1 | 1 | 1 | 1 | 1 | 1 | 1 | 1 |
| 65 | 2 | 2 | 1 | 1 | 1 | 1 | 1 | 1 | 1 | 2 | 2 | 2 |
| 67 | 4 | 4 | 4 | 4 | 4 | 8 | 4 | 4 | 4 | 8 | 8 | 4 |
| 69 | 4 | 4 | 4 | 1 | 1 | 1 | 1 | 1 | 8 | 4 | 4 | 4 |
| 73 | 4 | 4 | 4 | 4 | 4 | 4 | 4 | 4 | 4 | 8 | 4 | 8 |
| 74 | 8 | 8 | 8 | 4 | 4 | 8 | 4 | 4 | 4 | 8 | 8 | 4 |
| 75 | 2 | 4 | 2 | 2 | 2 | 2 | 2 | 2 | 2 | 4 | 4 | 4 |
| Vanc. | 1 | 2 | 1 | 4 | 2 | 2 | 1 | 0.5 | 2 | — | 4 | — |
| Dapt. | — | — | — | — | — | — | — | — | — | 1 | — | 1 |

Vanc.: Vancomycin
Dapt.: Daptomycin

### REFERENCES

1. Alkhaibari, I.; KC, H. R.; Angappulige, D. H.; Gilmore, D.; Alam, M. A., Novel pyrazoles as potent growth inhibitors of staphylococci, enterococci and Acinetobacter baumannii bacteria. Future medicinal chemistry 2022, 14, 233-244.
2. Alkhaibari, I. S.; Raj K. C., H.; Alnufaie, R.; Gilmore, D.; Alam, M. A., Synthesis of Chimeric Thiazolo-Nootkatone Derivatives as Potent Antimicrobial Agents. ChemMedChem 2021, 16, 2628-2637.
3. Hansa, R. K. C.; Khan, M. M. K.; Frangie, M. M.; Gilmore, D. F.; Shelton, R. S.; Savenka, A. V.; Basnaki. an, A. G.; Shuttleworth, S. L.; Smeltzer, M. S.; Alam, M. A., 4-4-(Anilinomethyl)-3-[4-(trifluoromethyl)phenyl]-1H-pyrazol-1-ylbenzoic acid derivatives as potent anti-gram-positive bacterial agents. European Journal of Medicinal Chemistry 2021, 219, 113402.
4. Saleh, I.; Raj Kc, H.; Roy, S.; Abugazleh, M. K.; Ali, H.; Gilmore, D.; Alam, M. A., Design, synthesis, and antibacterial activity of N-(trifluoromethyl)phenyl substituted pyrazole derivatives. RSC Medicinal Chemistry 2021, 12, 1690-1697.
5. Alnufaie, R.; Alsup, N.; Kc, H. R.; Newman, M.; Whitt, J.; Chambers, S. A.; Gilmore, D.; Alam, M. A., Design and synthesis of 4-[4-formyl-3-(2-naphthyl)pyrazol-1-yl]benzoic acid derivatives as potent growth inhibitors of drug-resistant Staphylococcus aureus. The Journal of Antibiotics 2020, 73, 818-827.

1. A thiazolylphenol compound, or a pharmaceutically acceptable salt thereof, wherein the phenol is optionally substituted with a second hydroxyl group and wherein the thiazolyl is substituted with—
one or more substituents independently selected from:
a phenylamino substituted at one or more positions with a halo, an alkyl, an alkoxyl, a haloalkyl, or any combination thereof,
an aryl optionally substituted at one or more positions with a halogen, an alkyl, a haloalkyl, a hydroxyl, an alkoxyl, cyano, oxo, carboxyl, acetamide, or any combination thereof,
an alkyl optionally substituted with a halo,
a heteroaryl optionally substituted with oxo, and
a biaryl or
two substituents forming a fused carbocyclyl with the thiozolyl.

2. The thiazolylphenol compound of claim 1, wherein the thiazolyl and phenol are covalently bound at the 4-position of the thiazolyl.

3. The thiazolylphenol compound of claim 2, wherein the phenol is

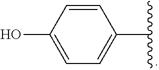 [SMILES: *c1ccc(O)cc1]

4. The thiazolylphenol compound of claim 2, wherein the phenol is

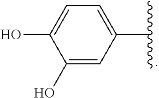 [SMILES: *c1ccc(O)c(O)c1]

5. The thiazolylphenol compound of claim 1, wherein the thiazolyl and phenol are covalently bound at the 2-position of the thiazolyl.

6. The thiazolylphenol compound of claim 5, wherein the phenol is

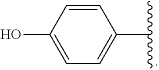 [SMILES: *c1ccc(O)cc1]

7. The thiazolylphenol compound of claim 5, wherein the phenol is

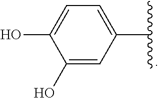 [SMILES: *c1ccc(O)c(O)c1]

8. The thiazolylphenol compound of claim 1, wherein the thiazolyl is substituted with one or more substituents selected from methyl, trifluoromethyl,

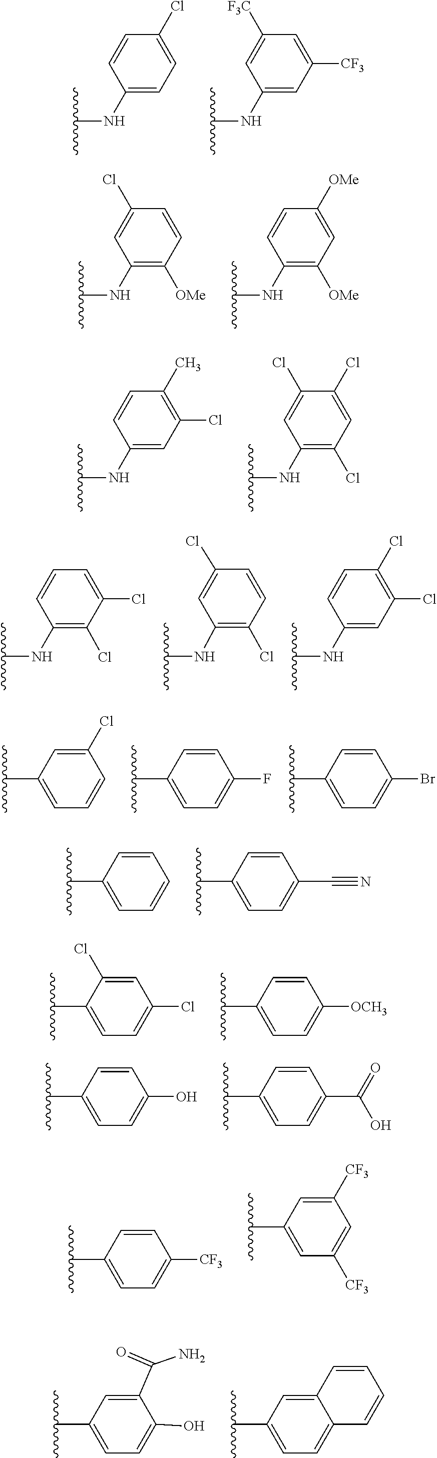

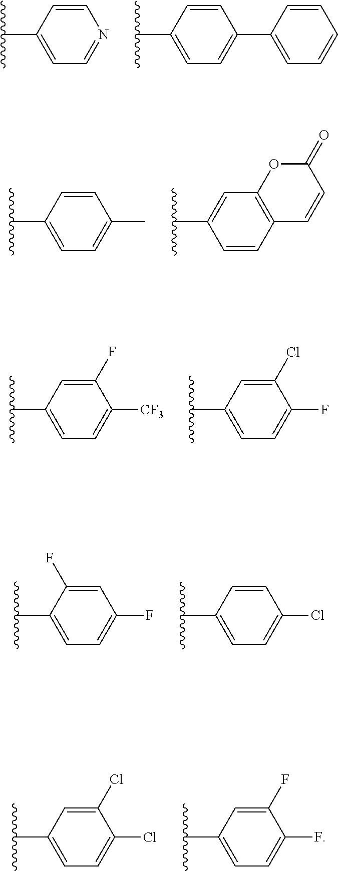

9. (canceled)

10. The thiazolylphenol compound of claim 1, wherein the thiazolylphenol compound is selected from:
4-(4-(trifluoromethyl)thiazol-2-yl)benzene-1,2-diol,
4-(4-phenylthiazol-2-yl)benzene-1,2-diol,
4-(4-(3-chlorophenyl)thiazol-2-yl)benzene-1,2-diol,
4-(4-(4-fluorophenyl)thiazol-2-yl)benzene-1,2-diol,
4-(4-(4-bromophenyl)thiazol-2-yl)benzene-1,2-diol,
4-(4-(2,4-dichlorophenyl)thiazol-2-yl)benzene-1,2-diol,
4-(4-(4-methoxyphenyl)thiazol-2-yl)benzene-1,2-diol,
4-(4-(4-hydroxyphenyl)thiazol-2-yl)benzene-1,2-diol,
4-(4-(4-(trifluoromethyl)phenyl)thiazol-2-yl)benzene-1,2-diol,
4-(4-(3,5-bis(trifluoromethyl)phenyl)thiazol-2-yl)benzene-1,2-diol,
4-(4-(pyridin-4-yl)thiazol-2-yl)benzene-1,2-diol,
4-(2-(3,4-dihydroxyphenyl)thiazol-4-yl)benzonitrile,
4-(2-(3,4-dihydroxyphenyl)thiazol-4-yl)benzoic acid,
4-(4-(naphthalen-2-yl)thiazol-2-yl)benzene-1,2-diol,
4-(4-([1,1′-biphenyl]-4-yl)thiazol-2-yl)benzene-1,2-diol,
5-(2-(3,4-dihydroxyphenyl)thiazol-4-yl)-2-hydroxybenzamide,
3-(2-(3,4-dihydroxyphenyl)thiazol-4-yl)-2H-chromen-2-one,
4-(4-(p-tolyl)thiazol-2-yl)benzene-1,2-diol,
4-(4-(4-fluoro-3-(trifluoromethyl)phenyl)thiazol-2-yl)benzene-1,2-diol,
4-(4-(4-fluorophenyl)-5-methylthiazol-2-yl)benzene-1,2-diol,
4-(4-(3-chloro-4-fluorophenyl)thiazol-2-yl)benzene-1,2-diol,
4-(5-methyl-4-phenylthiazol-2-yl)benzene-1,2-diol,
4-(5-methyl-4-(p-tolyl)thiazol-2-yl)benzene-1,2-diol,
4-(4-(3-chlorophenyl)-5-methylthiazol-2-yl)benzene-1,2-diol,
4-(4,5-diphenylthiazol-2-yl)benzene-1,2-diol,
4-(4-(2,4-difluorophenyl)thiazol-2-yl)benzene-1,2-diol,
4-(4-(4-chlorophenyl)thiazol-2-yl)benzene-1,2-diol,
4-(4,5,6,7-tetrahydrobenzo[d]thiazol-2-yl)benzene-1,2-diol,
4-(5,6-dihydro-4H-cyclopenta[d]thiazol-2-yl)benzene-1,2-diol,
4-(4,5-dihydronaphtho[1,2-d]thiazol-2-yl)benzene-1,2-diol,
4-(4-(3,4-dichlorophenyl)thiazol-2-yl)benzene-1,2-diol,
4-(4-phenylthiazol-2-yl)phenol,
4-(4-(4-bromophenyl)thiazol-2-yl)phenol,
4-(4-(2,4-dichlorophenyl)thiazol-2-yl)phenol,
4-(4-(naphthalen-2-yl)thiazol-2-yl)phenol,
4-(4-(3,5-bis(trifluoromethyl)phenyl)thiazol-2-yl)phenol,
4-(2-(4-hydroxyphenyl)thiazol-4-yl)benzoic acid,
4-(4-(3-chlorophenyl)thiazol-2-yl)phenol,
4-(4-(4-(trifluoromethyl)phenyl)thiazol-2-yl)phenol,
4,4′-(thiazole-2,4-diyl)diphenol,
4-(4-(3,4-dichlorophenyl)thiazol-2-yl)phenol,
4-(2-(4-bromophenyl)thiazol-4-yl)phenol,
4-(2-(3,5-dichlorophenyl)thiazol-4-yl)phenol,
4-(2-(4-(trifluoromethyl)phenyl)thiazol-4-yl)phenol,
4-(2-(4-bromophenyl)thiazol-4-yl)benzene-1,2-diol,
4-(2-(3,5-dichlorophenyl)thiazol-4-yl)benzene-1,2-diol,
4-{2-[4-(trifluoromethyl)phenyl]-1,3-thiazol-4-yl}benzene-1,2-diol,
4-(2-(4-chlorophenyl)thiazol-4-yl)phenol,
4-(2-(4-chlorophenyl)thiazol-4-yl)benzene-1,2-diol,
4-(2-(4-fluorophenyl)thiazol-4-yl)phenol,
4-(2-(4-fluorophenyl)thiazol-4-yl)benzene-1,2-diol,
4-(2-(3,4-difluorophenyl)thiazol-4-yl)phenol,
4-(2-(3,4-difluorophenyl)thiazol-4-yl)benzene-1,2-diol,
4-(2-(3-chlorophenyl)thiazol-4-yl)phenol,
4-(2-(3-chlorophenyl)thiazol-4-yl)benzene-1,2-diol,
4-(2-((4-chlorophenyl)amino)thiazol-4-yl)benzene-1,2-diol,
4-(2-((4-chlorophenyl)amino)thiazol-4-yl)phenol,
4-(2-((3,5-bis(trifluoromethyl)phenyl)amino)thiazol-4-yl)benzene-1,2-diol,
4-(2-((3,5-bis(trifluoromethyl)phenyl)amino)thiazol-4-yl)phenol,
4-(2-((3-methoxyphenyl)amino)thiazol-4-yl)benzene-1,2-diol,
4-(2-((5-chloro-2-methoxyphenyl)amino)thiazol-4-yl)phenol,
4-(2-((2,4-dimethoxyphenyl)amino)thiazol-4-yl)benzene-1,2-diol,
4-(2-((2,4-dimethoxyphenyl)amino)thiazol-4-yl)phenol,
4-(2-(3,4-dichlorophenyl)thiazol-4-yl)benzene-1,2-diol,
4-(2-(3,4-dichlorophenyl)thiazol-4-yl)phenol,
4-(2-((3-chloro-4-methylphenyl)amino)thiazol-4-yl)benzene-1,2-diol,
4-(2-((3-chloro-4-methylphenyl)amino)thiazol-4-yl)phenol,
4-(2-((2,4,5-trichlorophenyl)amino)thiazol-4-yl)benzene-1,2-diol,
4-(2-((2,4,5-trichlorophenyl)amino)thiazol-4-yl)phenol,
4-(2-((2,3-dichlorophenyl)amino)thiazol-4-yl)benzene-1,2-diol,
4-(2-((2,3-dichlorophenyl)amino)thiazol-4-yl)phenol,
4-(2-((2,5-dichlorophenyl)amino)thiazol-4-yl)benzene-1,2-diol,
4-(2-((2,5-dichlorophenyl)amino)thiazol-4-yl)phenol,
4-(2-((3,4-dichlorophenyl)amino)thiazol-4-yl)benzene-1,2-diol, and
4-(2-((3,4-dichlorophenyl)amino)thiazol-4-yl)phenol.

11. The thiazolylphenol compound of claim 1 having a formula 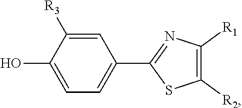
or a pharmaceutically acceptable salt thereof,
wherein R₃ is hydrogen or hydroxyl, and
wherein R₁ and R₂
are independently selected from hydrogen, the substituted or unsubstituted alkyl, the substituted or unsubstituted aryl, the substituted or unsubstituted heteroaryl, and the biaryl and both R₁ and R₂ are not hydrogen or
R₁ and R₂ together form a fused carbocyclyl with the thiozolyl.

12. The thiazolylphenol compound of claim 11, wherein R₃ is hydrogen.

13. The thiazolylphenol compound of claim 11, wherein R₃ is hydroxyl.

14. The thiazolylphenol compound of claim 1 having a formula 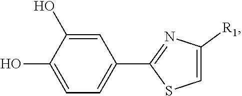
or a pharmaceutically acceptable salt thereof,
wherein R₁ is selected from the substituted or unsubstituted alkyl, the substituted or unsubstituted aryl, the substituted or unsubstituted heteroaryl, and the biaryl.

15. The thiazolylphenol compound of claim 11, wherein R₁ is selected from the substituted or unsubstituted aryl.

16. The thiazolylphenol compound of claim 1 having a formula 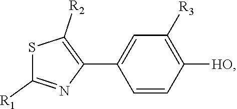
or a pharmaceutically acceptable salt thereof,
wherein R₃ is hydrogen or hydroxyl,
wherein R₂ is hydrogen, and
wherein R₁ is selected from the substituted phenylamino and the substituted or unsubstituted aryl.

17. The thiazolylphenol compound of claim 16, wherein R₃ is hydrogen.

18. The thiazolylphenol compound of claim 16, wherein R₃ is hydroxyl.

19. (canceled)

20. (canceled)

21. A pharmaceutical composition comprising the thiazolylphenol compound according to claim 1 and a pharmaceutically acceptable excipient, carrier, or diluent.

22. A method for the treatment of a subject in need of a treatment for an infection by a microbe, the method comprising administering an effective amount of the thiazolylphenol compound according to claim 1 or a pharmaceutical composition comprising the effective amount of the compound to the subject.

23. A method for inhibiting growth or proliferation or killing a microbe, the method comprising contacting the microbe with an effective amount of the thiazolylphenol compound according to claim 1.

24.-30. (canceled)
